(19) United States
(12) Patent Application Publication
(10) Pub. No.: US 2022/0160847 A1
(43) Pub. Date: May 26, 2022

(54) METHOD OF PREVENTING AND/OR TREATING A PLURALITY OF DISEASES

(71) Applicant: Mark Egly, Johnston, IA (US)

(72) Inventor: Mark Egly, Johnston, IA (US)

(21) Appl. No.: 17/527,730

(22) Filed: Nov. 16, 2021

Related U.S. Application Data
(60) Provisional application No. 63/116,200, filed on Nov. 20, 2020.

(51) Int. Cl. A61K 38/57; C12Q 1/6883

(52) U.S. Cl. CPC A61K 38/57; C12Q 2600/158; C12Q 1/6883

(57) ABSTRACT

A method of preventing and/or treating a plurality of diseases by determining if the patient is alpha-1 antitrypsin deficient by obtaining a biological sample from the patient and performing a genotyping assay on the biological sample to determine if the patient has a SERPINA1 alpha-1 antitrypsin deficient genotype or measuring the circulating level of alpha-1 antitrypsin in the sample. If the patient has a SERPINA1 alpha-1 antitrypsin deficient genotype or low levels of circulating alpha-1 antitrypsin, then alpha-1 antitrypsin is administered to the patient.

### CROSS-REFERENCE TO RELATED APPLICATIONS

This patent application claims priority to U.S. Provisional Patent Application No. 63/116,200 filed on Nov. 20, 2020, the entirety of which is incorporated herein fully by reference.

### FIELD OF THE DISCLOSURE

This disclosure relates generally to a method of treating a plurality of diseases with alpha-1 antitrypsin, and in particular to methods for diagnosing, preventing, and treating with alpha-1 antitrypsin, diseases and conditions caused by excessive neutrophils, neutrophil elastase, neutrophil extracellular traps, inflammation and/or oxidative stress.

### BACKGROUND OF THE DISCLOSURE

Alpha-1 antitrypsin (“AAT”) is a protein produced by the liver that protects the lungs from inflammation caused by infection and inhaled irritants. AAT is a member of the serine protease inhibitor superfamily known as Serpins. Alpha-1 antitrypsin deficiency, also known as AATD, Alpha-1, inherited emphysema, or genetic COPD, is a rare inherited condition that raises a person's risk for serious lung disease in adults or liver disease at any age. AATD occurs when a person's AAT is abnormal and/or cannot be released from the liver at the normal rate. This leads to a build-up of abnormal AAT in the liver that can cause liver disease, and decreased levels of AAT reaching the lungs which can lead to lung disease. There is no cure for AATD, but detection and treatment can help people with AATD manage symptoms and have better quality of life. AATD can be detected using genetic testing.

An orphan disease is defined as a condition that affects fewer than 200,000 people nationwide. With much competition for research dollars, diseases affecting larger numbers of people are prioritized, and research on orphan diseases frequently suffer from a lack of funding. With approximately 10,000 total people diagnosed and approximately 180 diagnosed each year, AATD is an orphan disease. As noted by Izaguirre Anariba et al, despite being a relatively common disease, AATD is frequently under-recognized with only approximately 15% of the population with AATD having been diagnosed with the disease, and knowledge of the disease within the medical community is limited. Some people with AATD are misdiagnosed with asthma.

The U.S. Food and Drug Administration has approved the use of four AAT products derived from human plasma: Prolastin, Zemaira, Glassia, and Aralast. These products for intravenous augmentation AAT therapy can cost up to $180,000 per year per patient. Nine hundred blood donations are needed to treat just one deficient patient per year at the present time. They are administered intravenously at a dose of approximately 60 mg/kg weekly in accordance with body weight of the patient to be infused, and administration takes approximately one hour with the patient's vitals taken before and after infusion. The half-life of AAT is 156 hours. Research and clinical trials into oral treatment, inhalers, and gene therapy are in progress. At this time, AAT is used mainly for treating AATD, fibromyalgia, and some dermatological issues. However, as a powerful inhibitor of neutrophils, neutrophil elastase, neutrophil extracellular traps, inflammation, and oxidative stress, AAT has the potential to treat many other conditions.

Neutrophils are a type of white blood cell that helps heal damaged tissues and resolve infections. Neutrophils play an important role in the body's immune response by killing and digesting bacteria and viruses. In her article entitled What are neutrophils and what do they do? Huizen describes the role of neutrophils in the body and typical neutrophil levels. Neutrophils play varied roles in the body, and the complex roles of neutrophils in homeostasis, immunity, and cancer were examined by Nicolas-Avila et al. While neutrophils play an important role in immune response, excessive neutrophils have been linked to inflammation leading to a number of diseases and conditions including emphysema (see Lucattelli et al), fibrosis (see Taooka et al), prehypertension and airflow limitation (see El-Eshmawy et al), vasculitis (see Jenne), and many others. As stated by Bardoel et al in their review of neutrophil function, “Neutrophils are endowed with a plethora of toxic molecules that are mobilized in immune responses. These cells evolved to fight infections, but when deployed at the wrong time and in the wrong place, they cause damage to the host.” As stated by Kovtun et al in their review describing the effects of severe trauma on the neutrophil phenotype and dysfunction and the consequences for tissue repair, “Alterations in neutrophil biology may contribute to trauma-associated complications, including immune suppression, sepsis, multiorgan dysfunction, and disturbed tissue regeneration. Furthermore, there is evidence that neutrophil actions depend on the quality of the initial stimulus, including trauma localization and severity, the micromilieu in the affected tissue, and the patient's overall inflammatory status.”

Neutrophil elastase is a serine proteinase released by activated neutrophils. The structure of the human neutrophil elastase gene is described in an article by Takahashi et al. As shown by Braga et al, Thymol shows promise as an inhibitor of inflammation associated with neutrophil elastase.

As stated by Korkmaz et al in their review of the physicochemical functions of neutrophil elastase, proteinase 3, and cathepsin G, “Neutrophil elastase, proteinase 3, and cathepsin G are three hematopoietic serine proteases stored in large quantities in neutrophil cytoplasmic azurophilic granules. They act in combination with reactive oxygen species to help degrade engulfed microorganisms inside phagolysosomes. These proteases are also externalized in an active form during neutrophil activation at inflammatory sites, thus contributing to the regulation of inflammatory and immune responses. As multifunctional proteases, they also play a regulatory role in noninfectious inflammatory diseases . . . . Because of their roles in host defense and disease, elastase, proteinase 3, and cathepsin G are of interest as potential therapeutic targets.”

Sahoo et al demonstrated “that the excessive recruitment of neutrophils to the site of infection causes tissue damage because of release of the protease elastase. Mice lacking neutrophil elastase have increased survival even though they carry an equal amount of bacteria in their organs as compared to the wild-type C57BL/6J. Thus, neutrophil elastase is a host defense mechanism that causes tissue damage and reduces host tolerance to infection.” In their review of the role of neutrophil elastase inhibitors in lung diseases, Polverino et al stated, “In many respiratory diseases characterized by an intense inflammatory response, the balance between proteolytic enzymes (proteases, including elastases) and their inhibitors (proteinases [or proteases] inhibitors) is not neutral. Excess activity of neutrophil elastase (NE) and similar proteases has been reported to cause tissue damage and to alter the remodeling process in many clinical conditions such as pneumonia, respiratory distress, and acute lung injury (ALI). Several experimental NE inhibitors have been tested in preclinical and clinical studies of different conditions of inflammatory lung injury such as ALI and pneumonia, with contrasting results.”

The human body has the capability of producing incredibly large amounts of neutrophil elastase and other neutrophils each day. Without proper inhibition of excessive neutrophils and neutrophil elastase, the body can easily enter a state of excess inflammation. Then, when these neutrophils are not properly used or inhibited, they can potentially transform a healthy body to unhealthy by multiple mechanisms, for instance by the formation of neutrophil extracellular traps that can become tumors that can potentially be malignant and metastasize throughout the body. A link between neutrophil extracellular traps (“NETs”) and several conditions, including sickle cell anemia, pre-eclampsia, artery problems, periodontitis, and coagulation problems was shown by Brinkmann and Zychlinsky.

It is not sufficient to be concerned with AATD solely as a stand-alone disease. We must be concerned about AAT's role in the overall health and wellness of patients. Over time, excessive neutrophil elastase and/or other neutrophils may form NETs and/or amyloid formations with the potential to eat away at healthy tissue, clog blood vessels, facilitate the growth of tumors and cysts, create inflammation in every part of the body, and transport through the circulatory system dangerous cells with the potential to spread throughout the body. Prevention of diseases including certain cancer, may be achieved with the introduction of adequate levels of AAT in advance of or at the onset of a neutrophil imbalance. Without proper inhibition, neutrophil elastase and all neutrophils can be a problem, but it is a problem that can be controlled, and problems potentially prevented and/or eliminated for many patients.

By the investigation of AAT levels and potential AATD we detect potentially severe liver disease. There is a significant fraction of people that may be harmed by many pharmaceuticals that clearly state they may cause liver damage as a side effect. Recognition that their condition may be caused by a liver condition would assist potential prevention of further damage, to an already damaged liver in those individuals.

A genetic predisposition, also known as a genetic susceptibility, is an increased likelihood of developing a particular disease due to the presence of one or more genetic factors, or a family history that indicates an increased risk of the disease. A genetic predisposition for a disease is not a diagnosis of the disease or a guarantee that a person will contract the disease. Knowledge of a genetic susceptibility can guide lifestyle choices or medical interventions. People can be aware of increased risk and know to seek early treatment if symptoms of the disease occur.

A carrier of a disease is a person who has inherited a recessive allele for a mutation associated with the disease. A carrier may show mild symptoms or none at all of the disease but is able to pass the allele onto their offspring, who may contract the disease if either parent, or both parents are carriers. A person with knowledge that he or she is a carrier of a disease can implement lifestyle choices or seek medical intervention for their children in an attempt to prevent or treat the disease and can at least be aware of the child's increased risk and seek early treatment if symptoms of the disease occur.

Tests may be ordered by a medical practitioner to determine if a patient has a genetic predisposition to a disease or is a carrier of the disease. In addition, many genetic testing services have emerged recently that allow consumers to directly purchase genetic profile reports based on a person's unique DNA. Companies such as 23andMe and Ancestry.com offer personalized reports that can indicate whether a person has a genetic predisposition to certain conditions or is a carrier of certain conditions.

Despite the advantages that could be realized by genetic testing to determine a person's genetic predispositions to disease or risk of passing a disease to offspring, some choose not to pursue genetic testing out of fear that the information revealed by their genetic profile could be used to their disadvantage. An example where genetic information could be damaging to a person is the insurance underwriting process. For example, while applying for a life insurance policy, the potential insured is asked a long series of intrusive questions regarding their physical and mental health, and in many cases a physical examination including extensive bloodwork is ordered. If the potential insured has received any medical care at all in recent years, their medical history is scrutinized, and insurance may be denied or offered at a higher premium due to increased risk. Genetic information is currently not included in the insurance underwriting process; however, for fear of being unable to obtain life insurance or of suffering other repercussions, many choose to remain ignorant of the genetic information that could improve their quality of life, improve their children's quality of life, or even prolong their lives. Knowledge of inherited conditions could impact not only the person seeking the genetic information, but also their living relatives and generations to come.

The Genetic Information Nondiscrimination Act of 2008 (“GINA”) is U.S. federal law that prohibits discrimination on the basis of genetic information with respect to health insurance and employment. However, GINA does not prevent third party vendors from sharing their information with insurance companies.

For the reasons stated above, and for other reasons which will become apparent to those skilled in the art upon reading and understanding the specification, there is a need in the art for an improved method for treating a plurality of diseases that considers the role of AAT in comorbid conditions.

Thus it is a primary object of the disclosure to provide a method for treating a plurality of diseases that prolongs and improves the quality of lives of individuals affected by AATD or non-AATD patients with lower levels of circulating AAT, or patients that do not fit the genetic profile of AATD and potentially suffer multiple comorbid conditions (for example: elderly that have proper Serpina1 alleles, but later in life produce inadequate amounts of AAT and develop conditions that we describe in this document such as, Alzheimer's and dementia that could benefit from higher levels of circulating AAT during their lifetimes).

Another object of the disclosure is to provide a method for treating a plurality of diseases that moderates neutrophils, serine proteases, inflammation, and oxidative stress.

Another object of the disclosure is to provide a method for treating a plurality of diseases that raises awareness of AATD, and non AATD comorbid conditions as well.

Another object of the disclosure is to provide a method for treating a plurality of diseases that incentivizes individuals, and medical providers to pursue genetic testing of patients for AATD and the testing of blood samples for circulating levels of AAT in order to improve quality of life and prolong lives.

Another object of the disclosure is to increase knowledge in the medical community of AATD and multiple conditions impacted by insufficient levels of circulating AAT.

Another object of the disclosure is to accelerate additional research of AATD and related conditions.

Another object of the disclosure is to accelerate additional research of non AATD conditions related to less than optimum levels of circulating AAT in the human body, that are previously unknown correlations to disease in patients, that would benefit from recognition of any correlation to related conditions of lower levels of circulating AAT.

Another object of the disclosure is to increase the production and use of AAT products, thus driving down the cost of AAT.

These and other objects, features, or advantages of the present disclosure will become apparent from the specification and claims.

### BRIEF SUMMARY OF THE DISCLOSURE

In one arrangement, the disclosure relates to a method of determining whether a patient is alpha-1 antitrypsin deficient.

In another arrangement, the disclosure relates to a method of determining whether a patient is suffering from non-AATD lower levels of circulating AAT.

In another arrangement, the disclosure relates to a method of controlling excess or runaway neutrophils such as neutrophil elastase with AAT.

In another arrangement, the disclosure relates to preventing, lessening the negative effects of, and treating Alzheimer's disease using alpha-1 antitrypsin (“AAT”).

In another arrangement, the disclosure relates to preventing, lessening the negative effects of, and treating abnormal amyloids, and myeloid cells using AAT.

In another arrangement, the disclosure relates to treating and/or preventing amyotrophic lateral sclerosis (“ALS” aka Lou Gehrig's Disease) using AAT.

In another arrangement, the disclosure relates to preventing, lessening the negative effects of, and treating anemia, and thrombocytopenia using AAT.

In another arrangement, the disclosure relates to preventing, lessening the negative effects of, and treating aneurysms using AAT.

In another arrangement, the disclosure relates to lessening and preventing antibiotic resistance using AAT.

In another arrangement, the disclosure relates to preventing or lessening the negative effects of Acute Respiratory Distress Syndrome (“ARDS”) caused by excess inflammation with AAT.

In another arrangement, the disclosure relates to preventing, lessening the negative effects of, and treating atherosclerosis using AAT.

In another arrangement, the disclosure relates to preventing, lessening the negative effects of, and treating a number of autoimmune diseases using AAT.

In another arrangement, the disclosure relates to preventing loss of lung function, and/or slowing the further loss of lung function, and/or slowing loss of lung tissue, while promoting the regeneration of lung tissue, including regeneration of alveoli in emphysema patients, COPD patients, those suffering from loss of lung function, and others suffering lung diseases, and/or lung damage using AAT and other protocols.

In another arrangement, the disclosure relates to preventing, lessening the negative effects of, and treating bone marrow disease using AAT.

In another arrangement, the disclosure relates to preventing, lessening the negative effects of, and treating certain cancers using AAT.

In another arrangement, the disclosure relates to preventing, eliminating, and lessening the negative effects of current cancer treatment side effects with AAT.

In another arrangement, the disclosure relates to preventing, lessening the negative effects of, and treating circulation problems using AAT.

In another arrangement, the disclosure relates to treating or preventing Cushing's disease using AAT.

In another arrangement, the disclosure relates to preventing, lessening the negative effects of, and treating the side effects of cystic fibrosis using AAT.

In another arrangement, the disclosure relates to reducing the size, area, and period of cytokine storms (also known as hypercytokinemia) in COVID-19 patients, other coronavirus patients, and pneumonia patients using AAT.

In another arrangement, the disclosure relates to preventing, lessening the negative effects of, and treating depression using AAT.

In another arrangement, the disclosure relates to preventing, lessening the negative effects of, and treating dementia using AAT.

In another arrangement, the disclosure relates to preventing, lessening the negative effects of, and treating diabetes using AAT.

In another arrangement, the disclosure relates to preventing, lessening the negative effects of, and treating conditions affecting the ears, and hearing using AAT.

In another arrangement, the disclosure relates to preventing, lessening the negative effects of, and treating eclampsia, preeclampsia, and hypertension using AAT.

In another arrangement, the disclosure relates to preventing, lessening the negative effects of, and treating endometriosis using AAT.

In another arrangement, the disclosure relates to preventing, lessening the negative effects of, and lessening the magnitude of seizures, and treating epilepsy using AAT.

In another arrangement, the disclosure relates to preventing, lessening the negative effects of, and treating GERD and other conditions affecting the esophagus and stomach using AAT.

In another arrangement, the disclosure relates to preventing, lessening the negative effects of, and treating conditions affecting the eyes and vision using AAT.

In another arrangement, the disclosure relates to preventing, lessening the negative effects of, and treating fibrosis using AAT.

In another arrangement, the disclosure relates to preventing, lessening the negative effects of, and treating heart disease using AAT.

In another arrangement, the disclosure relates to lessening the damage and the negative effects of a heart attack using AAT.

In another arrangement, the disclosure relates to preventing, lessening the negative effects of, and treating hemophilia using AAT.

In another arrangement, the disclosure relates to preventing and lessening the negative effects of the conditions of pulmonary hypertension, and prehypertension using AAT.

In another arrangement, the disclosure relates to preventing, lessening the negative effects of, and treating infertility, and sterility using AAT.

In another arrangement, the disclosure relates to preventing, lessening the negative effects of, and treating chronic inflammation using AAT.

In another arrangement, the disclosure relates to preventing, lessening the negative effects of, and treating kidney disease using AAT.

In another arrangement, the disclosure relates to preventing, lessening the negative effects of, and treating maternal hypoxia using AAT.

In another arrangement, the disclosure relates to preventing, lessening the negative effects of, and treating demyelination of cells related to multiple sclerosis using AAT.

In another arrangement, the disclosure relates to preventing, lessening the negative effects of, and treating muscular dystrophy using AAT.

In another arrangement, the disclosure relates to preventing, lessening the negative effects of, and treating neointimal lesions using AAT.

In another arrangement, the disclosure relates to maintaining and/or reducing of the size of, or eliminating cysts and tumors using AAT.

In another arrangement, the disclosure relates to preventing, treating, halting, or reducing the progression of IPMN cystic neoplasms and other cysts and tumors growing within the pancreas using AAT.

In another arrangement, the disclosure relates to preventing and lessening the negative effects of pain using AAT.

In another arrangement, the disclosure relates to preventing, lessening the negative effects, and treating of periodontal disease using AAT.

In another arrangement, the disclosure relates to a method of preventing, lessening the negative effects of, and treating pneumonia using AAT.

In another arrangement, the disclosure relates to preventing, reducing the size of, reducing the area of, and reducing the period of cytokine storms in the lungs and all vital organs.

In another arrangement, the disclosure relates to preventing, lessening the negative effects of the condition and/or treating psoriatic arthritis using AAT.

In another arrangement, the disclosure relates to preventing, lessening the negative effects of, and/or treating rheumatoid arthritis using AAT.

In another arrangement, the disclosure relates to preventing, lessening the negative effects of, and/or treating scoliosis, kyphosis, and other back issues using AAT.

In another arrangement, the disclosure relates to preventing, lessening the negative effects of, and/or treating sickle cell anemia using AAT.

In another arrangement, the disclosure relates to preventing, lessening the negative effects of, and/or treating strokes using AAT.

In another arrangement, the disclosure relates to using AAT in surgical applications.

In another arrangement, the disclosure relates to preventing, lessening the negative effects of, and/or treating syringomyelia using AAT.

In another arrangement, the disclosure relates to a method of preventing the destruction or degradation of thrombospondin1 within the lungs and throughout the human body and lessening the negative effects of other conditions that manifest without adequate levels of thrombospondin1, including protection against cancer, using AAT.

### BRIEF DESCRIPTION OF THE DRAWINGS

FIG. 1 depicts a device for at home testing for alpha-1 antitrypsin deficiency according to one embodiment.

FIG. 2 depicts a method for treating a condition according to one embodiment.

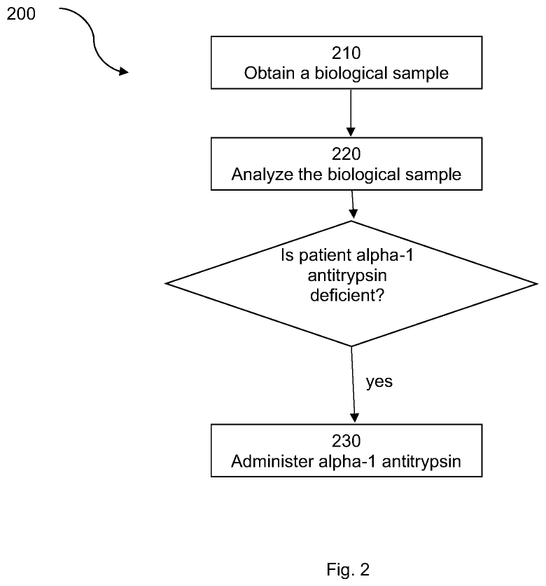

### DETAILED DESCRIPTION

In the following detailed description of the embodiments, reference is made to the accompanying drawings which form a part hereof, and in which is shown by way of illustration specific preferred embodiments in which the disclosure may be practiced. These embodiments are described in sufficient detail to enable those skilled in the art to practice the disclosure, and it is to be understood that other embodiments may be utilized, and that mechanical, procedural, and other changes may be made without departing from the spirit and scope of the present disclosures. The following detailed description is, therefore, not to be taken in a limiting sense, and the scope of the present disclosure is defined only by the appended claims, along with the full scope of equivalents to which such claims are entitled.

As used herein, the terminology such as vertical, horizontal, top, bottom, front, back, end and sides are referenced according to the views presented. It should be understood, however, that the terms are used only for purposes of description and are not intended to be used as limitations. Accordingly, orientation of an object or a combination of objects may change without departing from the scope of the disclosure.

As noted by Clemmensen et al in their paper describing AAT production, AAT is an important inhibitor of neutrophil proteases. Currently, AAT is used for treatment of emphysema and certain dermatological conditions. The present invention comprises and recognizes the discovery that AAT can be used to treat a number of diseases and conditions previously thought to be unrelated to AAT or AATD. As described by Blanco et al, AAT augmentation therapy has been approved for commercial use in selected adults with severe AATD-related pulmonary emphysema (i.e. PI*ZZ genotypes as well as combinations of Z, rare and null alleles expressing AAT serum concentrations less than 11 μmol/L), but AAT augmentation therapy has proven to be effective in a number of other conditions including as fibromyalgia, systemic vasculitis, relapsing panniculitis and bronchial asthma. For the purposes of this disclosure, AAT may be in any form or from any source (human, other mammal, plant based, or synthetic), and may be administered to a patient by any method such as injection, inhalation, intravenous administration, topical, gel, or oral administration. Administration may also be via a pump, similar to insulin pumps used by diabetics. Further the patient may be human or any other mammal.

AAT may be taken as a supplement, similar to other dietary supplements, to prevent or treat the diseases and conditions described in this disclosure through the serine protease inhibiting properties of AAT. AAT supplementation may be particularly beneficial to smokers, non-smokers who are exposed to secondhand smoke, and any person exposed to pollutants to counter the increased risk these individuals face from lung disease, cancer, and other conditions linked to inhalation of pollutants, as these pollutants are all known to destroy AAT in an individual with or without AATD. AAT supplements may be in the form of a tablet, powder, or liquid, and may be inhaled, infused, and/or taken in another manner to enhance the health and/or health outlook of an individual. With the production of medical grade AAT from human, plant and animal sources, AAT supplements can be made in an affordable manner. AAT may be taken as a supplement in conjunction with cannabidiol (“CBD”) to provide pain relief in addition to inflammatory and serine protease inhibiting properties of AAT.

In accordance with current treatment standards, AAT is administered to lung disease patients, and dosage for AAT augmentation is based on weight standards of such patients. Current treatment is based largely on what insurance companies will cover and pay and what current lung damage is. Supplies of AAT are limited as AAT is currently obtained from human blood donations. However, as Applicant's research has shown, AAT may be used to treat a much wider range of conditions, and the demand for AAT will increase. It is anticipated that larger quantities of AAT will be obtained through increased human blood donation and through animal blood, animal milk, or plant life. Going forward, proper AAT dosage should be based on circulating AAT levels rather than patient's weight alone.

As used in this disclosure, the terms condition or disease may be used interchangeably, and refer to any of the specific conditions described in this disclosure as benefitting from administration of AAT. Specifically the terms condition or disease may refer to any of the following conditions: Alzheimer's disease, abnormal amyloids or myeloid cells, amyotrophic lateral sclerosis (“ALS” aka Lou Gehrig's Disease), anemia, thrombocytopenia, aneurysms, antibiotic resistance, Acute Respiratory Distress Syndrome (“ARDS”), atherosclerosis, autoimmune diseases including but not limited to the specific autoimmune diseases listed in the section of this disclosure entitled “Autoimmune diseases”, loss of lung function, emphysema, COPD, lung disease, bone marrow disease, cancer including but not limited to the specific types of cancer listed in the section of this disclosure entitled “Cancer”, side effects of cancer treatment, circulation problems, Cushing's disease, cystic fibrosis, side effects of cystic fibrosis, cytokine storms, hypercytokinemia, coronaviruses including but limited to COVID-19, pneumonia, depression, dementia, diabetes, conditions affecting the ears and hearing, eclampsia, preeclampsia, hypertension, endometriosis, epilepsy, seizures, GERD, conditions affecting the esophagus or stomach, conditions affecting the eyes or vision, fibrosis, heart disease, heart attacks, hemophilia, pulmonary hypertension, prehypertension, infertility, sterility, inflammation, chronic inflammation, kidney disease, maternal hypoxia, multiple sclerosis, demyelinating diseases, muscular dystrophy, neointimal lesions, cysts, tumors, IPMN cystic neoplasms, pain, periodontal disease, cytokine storms in the lungs or other vital organs, psoriatic arthritis, rheumatoid arthritis, scoliosis, kyphosis, back or spine issues, sickle cell anemia, strokes, surgery, syringomyelia, or low thrombospondin1.

As used in this disclosure, the term treatment or treating refers to preventing, lessening the negative effects of, and/or treating a condition.

As used in this disclosure, administration of AAT may be in the form of a tablet, powder, or liquid, or may be inhaled, infused, and/or taken in another manner.

The administration of AAT, can protect the body from pollutants and secondhand smoke in addition to the potential for treating a plurality of diseases as described in this disclosure. For example, AAT may be made available to smokers, even those who are non-AATD, as a lung disease fighter in the form of an aerosol and pollutant fighting AAT product. Such a product may be available over the counter or by prescription. Thus, people who are exposed to secondhand smoke or other pollutants that destroy AAT in the body can prevent themselves from developing small cell lung cancer, other lung diseases, and other diseases mentioned in this disclosure.

Determining if a Patient is AAT Deficient or in the State of Having Low Circulating AAT without being AAT Deficient

A method 200 for treating a patient suffering from a condition described in this disclosure begin with determining if the patient is AAT deficient or in the state of having low circulating AAT without being AATD. Throughout this disclosure, AAT deficiency may mean either having an AATD genotype or having low levels of circulating AAT without having an AATD genotype.

Determining if the patient is AAT deficient may comprise and involve obtaining or having obtained a biological sample from the patient 210 and analyzing the biological sample 220. Analyzing the biological sample 220 may comprise performing or having involved performing a genotyping assay on the biological sample to determine if the patient is AAT deficient or determining if the patient has low circulating AAT without being AAT deficient by obtaining a quantitative measurement of the amount of AAT present in the biological sample.

Obtaining or having obtained a biological sample from the patient 210 may comprise and involve collecting in a collection device a blood sample, urine sample, saliva sample, or other biological sample from which the patient's circulating AAT level can be determined or from which AAT can be measured or from which nucleic acids and/or polypeptides can be isolated. The collection device may comprise specimen cups, swabs, glass slides, test tubes, lancets, tubes, syringes, vials, or any other device known in the art for collecting and/or storing a biological sample from which the patient's circulating AAT level can be determined or from which nucleic acids and/or polypeptides can be isolated.

Determining if the patient is AAT deficient further comprises analyzing the collected biological sample 220 to determine if the patient is AAT deficient or in the state of having low circulating AAT without being AAT deficient.

For example, the collected biological sample may comprise a sample of the patient's blood and determining if the patient is AAT deficient or has low AAT without being AATD may comprise measuring the circulating AAT level present in the blood sample. Alternatively, determining if the patient is AAT deficient may comprise performing or having performed a genotyping assay on the biological sample to determine if the patient is AAT deficient. The genotyping assay may detect which alleles of the Serpina1 gene are present in the biological sample, indicating whether the patient has a Serpina1 genotype indicative of AATD. The genotyping assay may detect if the patient has an AAT deficient genotype and/or phenotype directly or may detect characteristic mRNA of the polymorphic gene or its polypeptide expression product. Detection means suitable for use in the methods and devices of the present invention include those known in the art such as polynucleotides used in amplification, sequencing, and single nucleotide polymorphism (SNP) detection techniques, Invader® assays (Third Wave Technologies, Inc.), Taqman® assays (Applied Biosystems, Inc.), gene chip assays (such as those available from Affymetrix, Inc. and Roche Diagnostics), pyrosequencing, fluorescence resonance energy transfer (FRET)-based cleavage assays, fluorescent polarization, denaturing high performance liquid chromatography (DHPLC), mass spectrometry, and polynucleotides having fluorescent or radiological tags used in amplification and sequencing.

As described by Renoux et al, “AATD is an autosomal co-dominant disorder caused by mutations of the highly polymorphic SERPINA1 gene.” Codominance means that two different versions of the gene may be expressed, and both versions contribute to the trait. Renoux et al further state that, “The laboratory testing for this deficiency typically comprises serum alpha-1 antitrypsin quantification, phenotyping according to the isoelectric focusing pattern and genotyping if necessary.” To date approximately 132 SERPINA1 variants have been described and new genetic variants are frequently discovered. Since 2008, 22 new genetic variants of the SERPINA1 gene have been identified. Some mutations of the SERPINA 1 gene do not affect the production of AAT, while others cause AATD.

The most common allele of the SERPINA1 gene is referred to as M or PI*M. The M allele produces normal levels of AAT. Most people have two copies of the M allele (MM) in each cell. While people with the MM genotype are generally considered not at risk, there are some rare MM mutations that are now known to be associated with AATD and health problems. The contribution of rare SERPINA1 alleles must be considered when diagnosing AATD. A listing of known alleles of the SERPINA1 gene is shown in Table 1.

TABLE 1
Known Alleles of the SERPINA1 Gene

| | | Consequence |
|---|---|---|
| Allele | Pathogenic mutation | (SIFT and Polyphen scores) |
| Q0_{Faro} | c.-5 + 2dupT | AATD: protein absence |
| I | p.Arg39Cys | AATD: protein deficiency (0.03; 0.591) |
| M_{Malton} | p.Phe52del | AATD: protein deficiency (0; 0.99) |
| M_{Palermo} | p.Phe52del | AATD: protein deficiency (0; 0.99) |
| Q0_{Lisbon} | p.Thr68Ile | AATD: Protein absence (0; 0.98) |
| P_{Gaia} | p.Glu162Gly | AATD: protein deficiency (0.01; 0.80) |
| P_{Lowell} | p.Asp256Val | AATD: protein deficiency (0; 0.97) |
| Q0_{Gaia} | p.Leu263Pro | AATD: protein absence (0; 0.972) |
| T | p.Glu264Val | AATD: protein deficiency (0; 0.93) |
| Q0_{Oliveira do Douro} | p.Arg281Lysfs*17 | AATD: protein absence |
| [symbol] | p.Leu353Phefs*24 | AATD: protein absence |
| M_{Herleen} | p.Pro369Leu | AATD: protein deficiency (0; 0.99) |
| M_{Wurzburg} | p.Pro369Ser | AATD: protein deficiency (0; 0.99) |
| Q0_{Vila Real} | p.Met374Leufs*19 | AATD: protein absence |
| | p.Pro-23Leu | Possibly damaging (0.03; 0.50) |
| Z_{Wrexham} | p.Ser-19Leu | Not linked to AATD (0.05; 0.50) |
| | p.Trp-18Cys | Probably damaging (0.01; 0.91) |
| | p.Leu-13Met | Probably damaging (0; 0.99) |
| V_{Munich} | p.Asp2Ala | Not linked to AATD (0.06; 0.02) |
| | p.Pro3Leu | Benign (0.55; 0) |
| | p.His15Asn | Benign (0.41; 0) |
| | p.His16Arg | Benign (0.13; 0) |
| | p.Ala34Ser | Possibly damaging (0.06; 0.61) |
| M5_{Karlsruhe} | p.Ala34Thr | Not linked to AATD (0.13; 0.277) |
| Q0_{Knowloon} | p.Tyr38Ter | AATD: protein absence |
| M_{Procida} | p.Leu41Pro | AATD: protein deficiency (0.19; 0.99) |
| M_{Varallo} | p.Leu41-Phe51delfs*20 | AATD: protein deficiency |
| | p.His43Gln | Benign (0.35; 0.011) |
| M6_{Bonn} | p.Ser45Phe | Not linked to AATD (0; 0.867) |
| | p.Ser47Arg | Not linked to AATD (0.05; 0.01) |
| M_{Nichinan} | p.Phe52del | AATD: protein deficiency (0; 0.99) |
| S_{iiyama} | p.Ser53Phe | AATD: protein deficiency (0; 1) |
| M6_{Passau} | p.Ala60Thr | Not linked to AATD (0; 0.668) |
| | p.Leu66Arg | Probably damaging (002; 0.99) |
| M_{Mineral Springs} | p.Gly67Glu | AATD: protein deficiency (0:0.99) |
| | p.Thr72Ala | Possibly damaging (0; 0.82) |
| | p.Leu84Arg | Benign (0.02; 0.10) |
| Z_{Bristol} | p.Thr85Met | AATD: protein deficiency (0.02; 0.94) |
| Q0_{Ludwisghafen} | p.Ile92Asn | AATD: protein absence (0; 0.96) |
| Q0_{Soest} | p.Thr102Profs*10 | AATD: protein absence |
| Q0_{Devon} | p.Gly115Ser | AATD: protein deficiency (0.1; 0.97) |
| Q0_{Newport} | p.Gly115Ser | AATD: protein deficiency (0.1; 0.97) |
| Z_{Newport} | p.Gly115Ser | AATD: protein deficiency (0.1; 0.97) |
| | p.Leu120Phe | Possibly damaging (0; 0.87) |
| | p.Glu122Lys | Benign (0.15; 0.27) |
| | p.Lys129Glu | Probably damaging (0.07; 0.91) |
| | p.Phe130Leu | Probably damaging (0; 0.99) |
| | p.Ala142Asp | Probably damaging (0; 0.98) |
| V | p.Gly148Arg | Not linked to AATD (1; 0) |
| M2_{Obernburg} | p.Gly148Trp | Not linked to AATD (0.01; 0.70) |
| | p.Glu151Lys | Benign (0.27; 0.14) |
| Queen's | p.Lys154Asn | AATD: protein deficiency (0.06; 0.844) |
| Q0_{Chillichote} | p.Gln156Ter | AATD: protein absence |
| Q0_{Granite Falls} | p.Tyr160Ter | AATD: protein absence |
| Q0_{Bredevoort} | p.Tyr160Ter | AATD: protein absence |
| Q0_{amersfoort} | p.Tyr160Ter | AATD: protein absence |
| | p.Val161Met | Probably damaging (0; 0.99) |
| | p.Leu172Ser | Probably damaging (0; 0.99) |
| Q0_{Cork} | p.Thr180Serfs*11 | AATD: protein absence |
| | p.Lys193Ter | AATD: protein absence |
| Q0_{Trastevere} | p.Trp194Ter | AATD: protein absence |
| | p.Pro197His | Probably damaging (0; 0.98) |
| | p.Asp207Glu | Benign (0.09; 0.01) |
| | p.Val210Met | Possibly damaging (0; 0.86) |
| | p.Val216Met | Probably damaging (0; 0.99) |
| Q0_{Bellingham} | p.Lys217Ter | AATD: protein absence |
| F | p.Arg223Cys | Altered function (0.02; 0.52) |
| | p.Arg223His | Benign (0.09; 0.33) |
| P_{brescia} | Gly225Arg | AATD: protein deficiency (0.01; 0.95) |
| | p.Gly225Ala | Probably damaging (0; 0.97) |
| | p.Lys233Asn | Benign (0.14; 0) |
| Q0_{Perugia} | p.Val239Cysfs*3 | AATD: protein absence |
| P_{Duarte} | p.Asp256Val | AATD: protein deficiency (0; 0.97) |
| | p.Gly258Arg | Probably damaging (0; 0.99) |
| Q0_{Brescia} | p.Glu257Ter | AATD: protein absence |
| M_{Pisa} | p.Lys259Arg | AATD: protein deficiency (0; 0.958) |
| Q0_{Cairo} | p.Lys259Ter | AATD: protein absence |
| Q0_{Milano} | p.Lys259-Glu264delTer | AATD: protein absence |
| | p.His262Tyr | Benign (0.11; 0.06) |
| | p.His269Gln | Benign (0.31; 0.00) |
| | p.Leu276Pro | Probably damaging (0.03; 0.92) |
| | p.Asn278Ile | Benign (0.31; 0.00) |
| I_{Euskadi} | p.Arg281del | Not linked to AATD |
| | p.Ala284Ser | Benign (0.02; 0.15) |
| | p.Ile293Hisfs*6 | AATD: protein absence |
| Q0_{Torino} | p.Try297Ter | AATD: protein absence |
| | p.Asp298Glu | Benign (0.05; 0.08) |
| | p.Val302Ile | Not linked to AATD (1; 0.04) |
| Q0_{Cosenza} | p.Gln305Ter | AATD: protein absence |
| | p.Gly307Ser | Probably damaging (0; 0.97) |
| | p.Val311Ile | Benign (0.29; 0.07) |
| | p.Gly315Glu | Benign (1; 0.01) |
| Q0_{Hong Kong} | p.Leu318Serfs*17 | AATD: protein absence |
| Q0_{New Hope} | p.Gly320Glu | AATD: Protein absence (0; 0.98) |
| | p.Gly320Arg | Probably damaging (0.02; 0.99) |
| | p.Ala325Pro | Benign (0.26; 0.01) |
| QO_{Pordenone} | p.Leu327Argfs*12 | AATD: protein absence |
| S_{Munich} | p.Ser330Phe | Not linked to AATD (0; 0.89) |
| | p.Val333Met | Possibly damaging (0.08:0.53) |
| King's | p.His334Asp | AATD: protein deficiency (0.02; 0.943) |
| | p.Lys335Glu | Possibly damaging (0; 0.56) |
| W_{Bethesda} | p.Ala336Thr | AATD: protein deficiency (60%) (0; 0.93) |
| | p.Val337Gly | Probably damaging (0; 0.99) |
| | p.Val337Valfs*2 | AATD: protein absence |
| | p.Asp341Glu | Possibly damaging (0.03; 0.66) |
| P_{donauworth} | p.Asp341Asn | Not linked to AATD (0.06; 0.44) |
| P_{Saint albans} | p.Asp341Asn | Not linked to AATD (0.06; 0.44) |
| Z_{Augsburg} | p.Glu342Lys | AATD: protein deficiency (0.07; 0.99) |
| | p.Gly344Glu | Probably damaging (0.08; 0.99) |
| Q0_{Mattawa} | p.Leu353Phefs*24 | AATD: protein absence |
| M_{Pittsburgh} | p.Met358Arg | Altered function (0.24; 0.00) |
| | p.Met358Ile | Likely Altered function (0.25; 0.00) |
| L_{Offenbach} | p.Pro362Thr | Not linked to AATD (0; 0.10) |
| São Tomé | p.Pro362His | Not linked to AATD (0.04; 0.289) |
| Q0_{Bolton} | p.Pro362Argfs*12 | AATD: protein absence |
| Q0_{Clayton} | p.Pro362Profs*15 | AATD: protein absence |
| Q0_{Saarbruecken} | p.Pro362Profs*15 | AATD: protein absence |
| X_{Christchurch} | p.Glu363Lys | Not linked to AATD (0.74; 0.192) |
| E_{Taurisamo} | p.Lys368Glu | AATD: protein deficiency (0.01:0.083) |
| Q0_{Dublin} | p.Phe370Leufs*4 | AATD: protein absence |
| | p.Ile375Val | Benign (0.4; 0.00) |
| | p.Met385Val | Benign (1; 0.01) |
| Y_{Barcelona} | p.Pro391His | AATD: protein deficiency (0; 1) |
| Y_{orzinuovi} | p.Pro391His | AATD: protein deficiency (0; 1) |
| Q0_{Isola di Procida} | Del 17 Kb including | AATD: protein absence |
| | exons II-V | |
| Q0_{Riedenburg} | SERPINA1 deletion | AATD: protein absence |
| Q0_{Savannah} | g.5307_5308ins8bp | AATD: protein absence |
| Q0_{Porto} | c.-5 + 1G > A | AATD: protein absence |
| Q0_{Madrid} | c.-5 + 2dupT | |
| Q0_{Bonny blue} | c.-4 + 1Gdel | AATD: protein absence |
| Q0_{West} | c.-4 + 1G > T | AATD: protein absence |

The most common deficient allele associated with AATD is known as Z or PI*Z. The Z allele replaces the amino acid glutamic acid with the amino acid lysine at protein position 342 (written as Glu342Lys or E342K). This mutation results in a version of the SERPINA1 gene that produces very little AAT. People with the MZ genotype have a slightly increased level of impaired lung and/or liver function. While people with the MZ genotype are frequently thought of as being carriers only, a study by Beletic et al suggests that people with this genotype may be more than just carriers and have increased risk of conditions related to AATD. People with the SZ genotype (Glu264Val) have an increased risk of developing lung diseases such as emphysema, especially if they have other risk factors such as smoking. People with ZZ genotype are likely to have AATD. Gershagen and Janciauskiene describe a simple and accurate new ELISA-based test for identifying carriers of the AAT Z allele.

The second most common allele associated with AATD is known as S or PI*S. The S allele produces moderately low levels of AAT. Individuals with an MS or SS combination usually produce enough AAT to protect their lungs. People with the SZ combination have an increased risk of developing lung diseases such as emphysema, especially if they have other risk factors such as smoking, chemical exposure, and dust exposure.

In addition to determining a patient's genotype, AATD may be diagnosed by determining the concentration of AAT circulating in the patient's bloodstream. Determining if the patient is AAT deficient involves measuring what the actual circulating levels of AAT. This step is performed regardless of whether the patient has AATD or is non-AATD. Knowing circulating AAT even if the patient has a non-AATD genotype is important because diseases associated with low AAT levels can occur even when the patient does not have an AATD genotype. Currently, when a genotyping test does not come back with a bad allele, medical providers, insurance companies, and other companies do not test for circulating AAT.

Most hospital laboratories report serum AAT levels in milligrams per deciliter (mg/dL) with a reference range of approximately 100-300 mg/dL. Levels less than 80 mg/dL suggest a significant risk for lung disease. In addition to testing directly for AAT, AATD may be detected by measuring circulating levels of trypsin, neutrophil elastase, inhibitors, antinuclear antibodies, anti-smooth muscle antibodies in autoimmune hepatitis, antimitochondrial antibodies in primary biliary cirrhosis, iron studies in hemochromatosis, and ceruloplasmin in Wilson disease, and other substances that are inhibited or moderated by AAT. A testing device 160 for determining circulating levels of AAT or another substance in the patient's blood may use light, electricity, or chemical means for detecting AAT or the other substance.

Information regarding the patient's SERPINA1 genotype may be obtained from genetic testing performed either in a medical setting or by a private genetic testing company such as 23andMe or Geneology.com. The patient's SERPINA1 genotype may also be determined using a testing device 160.

Testing pregnant women for circulating ATT and AATD can be used to predict inherited AATD and related conditions in a developing fetus with the objective of preventing miscarriages, premature births, stillbirth, and premature death. Such prenatal screening or testing could be universal, with a stronger focus on mothers with family histories of liver disease, cancer, COPD, or emphysema, and mothers with other past or current problematic pregnancies and births. By detecting AATD and/or lower than standard circulating AAT levels in an expectant mother, the mother could be treated with AAT to increase the chances of a full term or longer-term pregnancy, increase the odds of a healthy birth and child, and avoid problems with higher blood pressure that could cause the rupturing of the delicate, fragile and immaturely developed blood vessels of the fetus. This type of rupturing has a history of high levels of loss of life of the baby in the neonatal period.

A carrier is an individual who carries and is capable of passing on a genetic mutation associated with a disease and may or may be entirely asymptomatic. Carriers are associated with diseases inherited as recessive traits. In order to have the disease, an individual must have inherited mutated alleles from both parents. An individual having one normal allele and one mutated allele may not have the disease. Two carriers may produce children with the disease.

Applicant's research shows that with the Serpina1 gene, patients with just one flawed gene are incapable of producing sufficient levels of AAT, even though the patient is not considered AATD. Thus, a patient with one flawed gene cannot produce the same levels of AAT circulating levels as a typical healthy person with two MM alleles. This finding leads to the conclusion that many more conditions exist that create a need for treatment with AAT. AATD carriers are not just carriers but have a strong susceptibility to AATD and to as many other diseases as those who have two flawed alleles and are diagnosed as having AATD. Applicant has encountered individuals having circulating AAT levels of 65 mg/dL who are suffering from comorbid conditions associated with insufficient AAT who are not classified as AATD because they have an M allele and no lung conditions, and as such are ineligible to receive AAT augmentation therapy because they do not have the paradigm illness recognized by the medical community. By current treatment standards and current limited supplies of AAT, only patients with severe lung disease are eligible to receive AAT augmentation therapy. By identifying individuals who are not classified as AATD but have low circulating AAT levels, non-lung and non-liver comorbidities are identified and many conditions may be prevented, lessened, and/or treated for patients without lung or liver disease.

The method 200 proceeds to step 230 where, if the patient is AATD or has low circulating AAT without being AATD, then AAT is internally administered to the patient such that the patient's total alpha-1 antitrypsin level is in the range of 100-300 mg/dL. AAT dosage should be calculated as a function of the current level of circulating AAT in the patient plus the additional amount of AAT required to maintain the patient within the standard range of MM allele individuals. This range typically, is described as being, between 100-300 mg/dL. Previously AATD patients have only been brought up to the lower end of this range. Dosages should be made available every 6 to 7 days as the life of AAT, according to Prolastin-C maker Grifols, is 156 hours, which is approximately 6.5 days. Typical augmentation therapy dosage for AATD patients is 60 mg/kg, but physicians have discretion to prescribe AAT based on current circulating levels of AAT. By correlating the typical dosage to circulating AAT levels currently in individuals, this becomes an alternative method to mathematically calculate dosages of AAT for patients with the conditions described within this disclosure. Currently Kamada is the producer of the product Glassia. Glassia has been in trial to treat patients with AAT in the form of an aerosol applied with a nebulizer at a current rate of 80 mg twice daily for a total of 160 mg per day. Kamada received FDA approval in 2019.

Acute Respiratory Distress Syndrome

Acute respiratory distress syndrome (“ARDS”) occurs when lungs are severely injured, and may be caused by sepsis, pneumonia, trauma, blood transfusions, pancreatitis, near drowning, inhaling harmful substances, or other lung injury. ARDS causes fluid to leak into the lungs, making it difficult for oxygen to enter the bloodstream.

The American Thoracic Society has published that Neutrophil Elastase (“NE”) can cause the neutralization of other neutrophils' ability to fight off bacteria. As documented by McCarthy et al, AAT inhibits neutrophil elastase (“NE”) in the lungs. AAT also inhibits other neutrophil-derived proteases. Thus, AAT protects lung tissue from degradation. There is a significantly higher burden of neutrophils in the lungs of individuals with AATD compared with healthy individuals, and the significant neutrophil burden contributes to increased proteolytic activity and inflammation. Further, NE impairs the ability of other neutrophils to kill bacteria within the lungs.

NE and its relationship to cytokine storms plays a role in ARDS. A cytokine storm occurs when there is an excessive and uncontrolled release of pro-inflammatory cytokines. It can result in acute respiratory distress syndrome (“ARDS”), multiple organ failure, and death. Cytokine storms have been seen in severe SARS, MERS, and COVID-19 cases. See Mangalmurti. Clinical reports from China published by Zhang et al. in the journal Clinical Immunology (Zhang et al 2020), revealed that, in addition to lymphocytopenia, COVID-19 patients had high inflammatory parameters and proinflammatory cytokines, particularly IL2, IL6, IL7, IL8, IL10, and TNFa. Inflammatory cytokines were further elevated in patients admitted to the ICU7, all suggestive of a COVID-19 induced cytokine storm. Autopsy reports from COVID-19 patients found atrophy in the spleen and secondary lymphoid tissues. Since these organs do not express ACE2, the receptor for COVID-19 infection, Zhang et al. theorize that damage to the immune system was a result of a cytokine storm. Zhang et al. conclude, in agreement with Dr. Roth, that in light of the real possibility of a cytokine storm in critically ill patients, an anti-inflammatory treatment may be necessary, but that “a timely anti-inflammation treatment initiated at the right window of time is of pivotal importance and should be tailored in individual patients to achieve the most favorable effects.” Treatment of cytokine storms by timely administration of an anti-inflammatory agent is further discussed in Kimberly B. Bjugstad's article.

Due to the link between AATD and ARDS, in one embodiment AAT may be used to treat, control, or prevent ARDS in patients, either with or without known AATD. A diagnosis of ARDS may prompt a medical provider to test a patient for AATD. Alternatively, a patient diagnosed with ARDS may seek genetic testing for AATD through an independent genetic testing company such as 23andMe or geneology.com, a pharmaceutical company supplied test kit or other private methods available.

A method for treating a patient suffering from ARDS with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended. AAT may be administered in the form of a powder, tablet, or liquid, or AAT may be inhaled, infused, or taken in another manner. When treating cytokine storms, direct inhalation of AAT into the lungs in varying dosages can be found to be extremely important, as used by the product Glassia from the company Kamada in treating the condition. In the case of treating pneumonia, medical providers may administer AAT more frequently as neutrophil-lymphocyte count ratios (NLCR) can be monitored in these emergency situations hourly.

Through the administration of AAT to a patient suffering from ARDS, the administered AAT inhibits NE that contributes to ARDS; thus curing, controlling, or reducing the severity of ARDS. By using AAT to control NE, ARDS may be controlled or prevented before it ever develops; therefore, reducing or eliminating the need to use antibiotics to treat ARDS and reduces bacteria from becoming antibiotic resistant.

Alzheimer's Disease

Excessive neutrophil elastase (“NE”) is associated with Alzheimer's disease. Alzheimer's disease, commonly known simply as “Alzheimer's,” is a progressive disorder that causes brain cells to degenerate and die, also known as demyelination. Patients suffering from Alzheimer's disease experience memory loss and loss of cognitive function.

Inflammation is a hallmark of Alzheimer's disease. Excess serine proteases such as NE, cathepsin G, proteinase 3, etc., have been shown to contribute to disease pathogenesis by their buildup and formation of lesions in the brain. Nielsen et al showed that Alzheimer's disease is characterized by inflammation which may be controlled by serine protease inhibitors such as AAT. When bacterial or viral control is not necessary, as in the case of Alzheimer's disease, serine protease activity of neutrophils is unnecessary, and neutralization of excess serine proteases is indicated to eliminate inflammation in the most effective and efficient manner. AAT acts as an inhibitor of NE. Alzheimer's disease can be reversed through the inhibition of neutrophil adhesion where neutrophil extracellular traps can become present or are currently present. Administration of AAT to Alzheimer's patients can slow or stop the advancement of Alzheimer's and can result in improved memory and cognitive function in some patients. In some cases, the human body stops or slows production of AAT as age progresses. Dementia and Alzheimer's disease can manifest itself because pollutants and smoke destroy AAT over time. AAT can break the blood brain barrier where NE freely flows, therefore allowing the damaging process of the demyelination process of the brain cells to be halted by the attacking, free flowing NE. Oxidative stress contributes to the development of Alzheimer's disease. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of Alzheimer's disease through suppression of oxidative stress.

The cerebral microcapillary endothelium forms a highly important barrier between the blood and the interstitial fluid of the brain (blood-brain barrier) that controls the passage of molecules and cells in and out of the central nervous system. A study by von Wedel-Parlow et al demonstrates that neutrophils cross the blood brain barrier primarily on transcellular pathways. As shown by Joice et al, resting neutrophils induce acute reductions in permeability of the blood brain barrier. Moxon-Emre and Schlichter demonstrated that neutrophil depletion reduces breakdown of the blood brain barrier, axon injury, and inflammation after intracerebral hemorrhage. As shown by Liu et al, neutrophils play roles in blood flow, edema, hypoxia, neuroinflammation, and neurodegeneration linked to traumatic brain injury, and patients suffering from past traumatic brain injury are more prone to developing Alzheimer's disease.

As a powerful inhibitor of NE, inflammation, and oxidative stress, in one embodiment AAT may be used to treat, control, or prevent Alzheimer's in patients, either with or without known AATD. A diagnosis of Alzheimer's or dementia may prompt a medical provider to test a patient for AATD or for circulating AAT levels in non AATD patients. Alternatively, a patient diagnosed with Alzheimer's or dementia may seek genetic testing for AATD through an independent genetic testing company such as 23andMe or geneology.com, a pharmaceutical company supplied test kit or other private methods available.

A method for treating a patient suffering from Alzheimer's with AAT begins with the step of determining if the patient is AAT deficient or has lower AAT levels but is non-AATD. Determining if the patient is AAT deficient involves measuring circulating levels of AAT are within the patient. This step is performed regardless of whether the patient has AATD or is non-AATD. Determining if the patient is AAT deficient or has lower AAT levels but is non-AATD, proceeds as previously described in this disclosure in the section entitled “Determining if a patient is AAT deficient or in the state of having low circulating AAT without being AAT deficient.” By testing for low levels of AAT, it can be determined whether uncontrolled and over abundant NE in the body may be a factor in the buildup of amyloid-β (Aβ) deposits and lesions in the brain, contributing to the development of Alzheimer's disease. If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from Alzheimer's, the administered AAT neutralizes NE; thus curing, controlling, or reducing the severity of the patient's Alzheimer's while at the same time offering protection from other conditions listed within this disclosure. A method of treating an individual suffering from Alzheimer's by administering to the individual an effective amount of AAT.

Amyloids and Myeloid Cells

Amyloids are aggregates of proteins characterized by a particular morphology. Amyloids have been linked to a number of diseases. Pathogenic amyloids form when previously healthy proteins lose their normal structure and function and form fibrous deposits in plaques around cells which can disrupt the healthy function of tissues and organs.

Myeloid cells arise from myeloid progenitor cells and will eventually become the specific adult blood cells: basophils, neutrophils, eosinophils, monocytes, macrophages, erythrocytes, and platelets. Upon pathogen invasion, myeloid cells are rapidly recruited into local tissues where they are activated for phagocytosis as well as secretion of inflammatory cytokines, thereby playing major roles in innate immunity. Genetic alterations in myeloid cells may cause an abnormal increase in mature myeloid or blast cells resulting in chronic or acute myelogenous leukemia.

Patients with pathogenic amyloids or abnormal myeloid cells may benefit from the administration of AAT regardless of whether the patient has known AATD. A diagnosis of pathogenic amyloids or abnormal myeloid cells may prompt a medical provider to test for AATD. Alternatively, a patient diagnosed with pathogenic amyloids or abnormal myeloid cells may seek genetic testing for AATD through an independent genetic testing company such as 23andMe or geneology.com, a pharmaceutical company supplied test kit or other private methods available.

A method for treating a patient suffering from pathogenic amyloids or abnormal myeloid cells or a condition associated with these conditions with AAT begins with the step of determining if the patient is AAT deficient or has lower AAT levels but is non-AATD. Determining if the patient is AAT deficient or in the state of having low circulating AAT without being AAT deficient, proceeds as previously described in this disclosure in the section entitled “Determining if a patient is AAT deficient or in the state of having low circulating AAT without being AAT deficient.”

If the genotyping assay obtained from determining AAT deficiency indicates that a patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended

Through the administration of AAT, pathogenic amyloids or abnormal myeloid cells are controlled; thus curing, controlling, or reducing the severity of the patient's symptoms.

Amyotrophic Lateral Sclerosis (“ALS” Aka Lou Gehrig's Disease)

Excessive neutrophil elastase (“NE”) is associated with amyotrophic lateral sclerosis (“ALS”), which is commonly known as Lou Gehrig's disease. ALS is a progressive and fatal neurodegenerative disease leading to muscle weakness and paralysis. Neuroinflammation is a recognized pathogenic mechanism underlying the motor neuron degeneration that occurs in ALS. Trias et al demonstrated that degranulating mast cells were abundant in the quadriceps muscles of ALS subjects but not in controls. Additionally, Trias et al found that mast cells and neutrophils were abundant around motor axons in the extensor digitorum longus muscle, sciatic nerve, and ventral roots of symptomatic SOD1G93A rats, indicating that immune cell infiltration extends along the entire peripheral motor pathway. Therefore, there is evidence for a contribution of immune cells in peripheral motor pathway degeneration that can be therapeutically targeted by tyrosine kinase inhibitors.

Excessive immune cells associated with degeneration in ALS patients may be targeted with the administration of AAT regardless of whether or not the patient has known AATD. A diagnosis of ALS may prompt a medical provider to test a patient for AATD. Alternatively, a patient diagnosed with ALS may seek genetic testing for AATD through an independent genetic testing company such as 23andMe or geneology.com, a pharmaceutical company supplied test kit or other private methods available.

A method for treating a patient suffering from ALS begins with the step of determining if the patient is AAT deficient or suffering from lower levels of circulating AAT. Determining if the patient is AAT deficient or suffering from lower levels of circulating AAT proceeds as previously described in this disclosure in the section entitled “Determining if a patient is AAT deficient or in the state of having low circulating AAT without being AAT deficient.” If the genotyping assay finds a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT, ALS may be prevented, controlled, or reversed, ALS symptoms may be relieved, and the inflammation and pain associated with ALS may be reduced or eliminated. Additionally, with AAT therapy, lung functions and difficulty breathing may improve, and may also potentially improve other previously unassociated comorbidities.

Anemia and Thrombocytopenia

Iron deficiency anemia is a condition in which blood lacks adequate healthy red blood cells, which carry oxygen to the body's tissues. Iron deficiency anemia occurs due to insufficient iron. Iron deficiency anemia may leave the patient tired and short of breath.

Aplastic anemia is a condition that occurs when a patient's body stops producing enough new blood cells. The condition leaves the patient fatigued and more prone to infections and uncontrolled bleeding.

Thrombocytopenia is a condition characterized by a low blood platelet count. Platelets, also called thrombocytes, are colorless blood cells that help blood clot. Platelets stop bleeding by clumping and forming plugs in blood vessel injuries. Thrombocytopenia often occurs because of a separate disorder, such as leukemia or an immune system problem, or it can be a side effect of taking certain medications.

In one embodiment, AAT may be used to treat, control, or prevent anemia and thrombocytopenia in patients, either with or without known AATD. A diagnosis of anemia or thrombocytopenia may prompt a medical provider to test a patient for AATD. Alternatively, a patient diagnosed with anemia or thrombocytopenia may seek genetic testing for AATD through an independent genetic testing company such as 23andMe or geneology.com, a pharmaceutical company supplied test kit or other private methods available.

A method for treating a patient suffering from anemia or thrombocytopenia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals.

This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. Thus, the patient's anemia or thrombocytopenia is cured or controlled, or the severity is reduced.

Aneurysms

An aneurysm is a ballooning of a weakened area in an artery. Aneurysms often occur in the aorta, brain, back of the knee, intestine, or spleen. A ruptured aneurysm can result in internal bleeding, stroke, and can be fatal. Aneurysms often have no symptoms until they rupture. Typical treatment of an aneurysm varies from monitoring to emergency surgery, depending on the location of the aneurysm.

In adults, aneurysms typically develop in patients with risk factors which have affected their blood vessels over the course of many years, such as smoking, hypertension, long-term excessive alcohol intake and aging. These factors are not present in pediatric patients, and pediatric aneurysms are very rare. It is estimated that 5 to 10 percent of pediatric aneurysms are related to head trauma, approximately 15 percent are the result of infection, and up to 33 percent are associated with underlying conditions such as neurofibromatosis type I, connective tissue disorders (Marfan syndrome or the vascular subtype Ehlers-Danlos syndrome), polycystic kidney disease, sickle cell anemia or malformation of the blood vessels. Many pediatric aneurysms arise for reasons that are not yet understood.

As shown by Tutino et al, RNA expression from circulating neutrophils is associated with intracranial aneurysms. Due to AAT's ability to control neutrophil levels, AAT may be used for the treatment of unruptured aneurysms. A diagnosis of an aneurysm may prompt a medical provider to test a patient for AATD or for low levels of circulating AAT. Alternatively, a patient diagnosed with an aneurysm may seek genetic testing for AATD through an independent genetic testing company such as 23andMe or geneology.com, a pharmaceutical company supplied test kit or other private methods available.

A method for treating a patient suffering from an aneurysm with AAT begins with the step of determining if the patient is AAT deficient or has lower AAT levels without being AATD. Determining if the patient is AAT deficient proceeds as previously described in this disclosure in the section entitled “Determining if a patient is AAT deficient or in the state of having low circulating AAT without being AAT deficient.” If the genotyping assay indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as between 100 mg/dL and 300 mg/dL. Previous AAATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days.

Through the administration of AAT to a patient suffering from an aneurysm, the administered AAT decreases levels of neutrophils; thus curing, controlling, or reducing the severity of the patient's aneurysm.

Atherosclerosis

Atherosclerosis is the buildup of fats, cholesterol, and other substances on artery walls. Such buildup can obstruct blood flow. Additionally, plaques may rupture, causing acute occlusion of an artery. Atherosclerosis often has no symptoms until a plaque ruptures or the buildup is severe enough to block blood flow. A healthy diet and exercise can help. Treatments include medications, procedures to open blocked arteries, and surgery.

Oxidative stress contributes to the development of cancer. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of atherosclerosis through suppression of oxidative stress.

Due to the link between atherosclerosis and AAT, in one embodiment AAT may be used to treat, control, or prevent atherosclerosis in patients, either with or without known AATD. A diagnosis of atherosclerosis may prompt a medical provider to test a patient for AATD. Alternatively, a patient diagnosed with atherosclerosis may seek genetic testing for AATD through an independent genetic testing company such as 23andMe or geneology.com, a pharmaceutical company supplied test kit or other private methods available.

A method for treating a patient suffering from atherosclerosis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days.

Physicians may fluctuate on 5 to 8 days between treatments, or every 6 to 7 days for typical treatments when dosages are recommended

Through the administration of AAT to a patient suffering from atherosclerosis, the patient's atherosclerosis may be cured or controlled, or the severity may be reduced.

Autoimmune Diseases

A large number of autoimmune diseases occur because the body's homeostasis is altered by an overabundance of neutrophils or neutrophil elastase (“NE”). The level of neutrophils in the body can be controlled through administration of or supplementation with AAT. A diagnosis of an autoimmune disease may be indicative of AATD or an insufficient level of AAT without being AATD. By identifying lower or deficient AAT levels improved disease control can be realized through administration of or supplementation with AAT. In addition, patients will experience less inflammation as a result of the administration of, or supplementation with AAT. With reduced inflammation, need for aspirin and similar products to relieve pain will be reduced and potential liver disease and other conditions associated with overuse of such pain relievers will be reduced.

Autoimmune diseases that may be prevented, cured, or reduced in severity by administration or supplementation with AAT include: acute disseminated encephalomyelitis (see Saadoun et al 2011), Addison's disease, adiposis dolorosa, adult-onset Still's disease (see Mitrovic and Fautrel), alopecia areata, ankylosing spondylitis (see Muley et al), anti-glomerular basement membrane nephritis, anti-neutrophil cytoplasmic antibody-associated vasculitis, anti-N-methyl-D-aspartate receptor encephalitis (see Luca et al), antiphospholipid syndrome (see Yalavarthi et al), Antisynthetase syndrome, aplastic anemia, autoimmune angioedema, autoimmune enteropathy, autoimmune hemolytic anemia, autoimmune hepatitis, Cogan's syndrome or autoimmune inner ear disease (see Greco et al and Wiesner et al), autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy (see Hooks et al), autoimmune thrombocytopenic purpura (see Mikes et al), autoimmune thyroiditis, autoimmune urticaria (see Sabroe et al), autoimmune uveitis (see Polanska et al), Balo concentric sclerosis, Behcet's disease (see Sun & Yang), bullous pemphigoid (see Liu et al), Celiac disease, chronic fatigue syndrome, chronic inflammatory demyelinating polyneuropathy, Churg-Strauss syndrome (see Drage et al), Cicatrical pemphigoid (see Liu et al), cold agglutinin disease, complex regional pain syndrome, Crest syndrome or limited scleroderma, Crohn's disease, dermatitis herpetiformis (see Gornowicz-Porowska et al), dermatomyositis, diabetes mellitus type 1, discoid lupus erythematosus, endometriosis, Enthesitis, Enthesitis-related arthritis, eosinophilic esophagitis (see Cheng et al), eosinophilic fasciitis (see Islam et al), epidermolysis bullosa acquisita (see Yu et al), d, essential mixed cryoglobulinemia (see Laidlaw et al), Evans syndrome, fibromyalgia, gastritis (see Nakata et al), gestational pemphigoid (see Varraes et al), giant cell arteritis (see Cats et al), Graves disease, Graves ophthalmopathy, Guillain-Barre syndrome (see Shen et al), Hashimoto's encephalopathy (see Huang et al), Hashimoto thyroiditis, Henoch-Schonlein purpura, hidradenitis suppurativa (see Lapins et al), idiopathic inflammatory demyelinating diseases (see Dunham et al), IgG4-related disease (see Umehara et al), inclusion body myositis (see Keller et al), inflammatory bowel disease also known as IBD (see Wera et al), intermediate uveitis (see Kuromitsu et al), interstitial cystitis (see Kuromitsu et al), juvenile arthritis (see Kaplan 2013), Kawasaki's disease (see Takeshita et al), Lambert-Eaton myasthenic syndrome, leukocytoclastic vasculitis, lichen planus, lichen sclerosis, ligneous conjunctivitis, linear IgA disease (see Chaudhari & Mobini), lupus (see Garcia-Romo et al), lupus nephritis (see Sule et al), lupus vasculitis (see Kaplan 2012), Lyme disease, Meniere's Disease, microscopic colitis (see Berthold et al), microscopic polyangiitis, microscopic polyangiitis also known as MPA, mixed connective tissue disease (see Hau et al), Mooren's ulcer (see Shoaib), morphea, Mucha-Habermann disease, multiple sclerosis, myasthenia gravis (see Yang et al), myocarditis (see Bracamonte-Baran & Cihikova), myositis (see Zhang et al), neuromyelitis optica (see Saadoun et al 2012), optic neuritis (see Bisgaard et al), Ord's thyroiditis, palindromic rheumatism, paraneoplastic cerebellar degeneration (see Afzal et al), Parry-Romberg syndrome (see Karukayil et al), Parsonage-Turner syndrome, pediatric autoimmune neuropsychiatric disorder associated with streptococcus, pemphigus vulgaris (see Hoss et al), pernicious anemia (see Jones), Pityriasis lichenoides et Varioliformis acuta (see Liu, Yi-Di et al), Poems Syndrome (see Nozza), polyarteritis nodosa, polymyalgia rheumatica (see Iwata & Mizuno), polymyositis (see Ha et al) Postmyocardial infarction syndrome (see Horckmans et al), Postpericardiotomy syndrome (Sevuk et al), primary biliary cirrhosis (see Huang 2016), primary sclerosing cholangitis (see Duerr et al), progressive inflammatory neuropathy, psoriasis, psoriatic arthritis (see Macleod et al), pure red cell aplasia, pyoderma gangrenosum (see Dwarakanath et al), Raynaud phenomenon, reactive arthritis (see Lauhio et al), relapsing polychondritis (see Jasin & Taurog), restless leg syndrome, retroperitoneal fibrosis, rheumatic fever, rheumatoid arthritis (see Ottonello et al), rheumatoid vasculitis, sarcoidosis (see Golubicic et al), Schnitzler syndrome, scleroderma, Sjogren's syndrome, stiff person syndrome, subacute bacterial endocarditis, Susac's Syndrome, sydenham chorea, sympathetic ophthalmia (see Arevalo et al), systemic lupus erythematosus, systemic scleroderma, thrombocytopenia (see Mikes et al), Tolosa-Hunt syndrome, transverse myelitis, ulcerative colitis (see Berthold et al), undifferentiated connective tissue disease, urticaria, urticarial vasculitis, vasculitis, vitiligo (see Donadieu et al).

In particular, Hokari et al indicated that dense neutrophil infiltration is one of the characteristic pathological findings in the inflamed mucosa of ulcerative colitis patients, and several proteinases derived from neutrophils have been reported to be involved in the pathology of inflammatory bowel disease.

Due to the link between autoimmune disease and AAT, in one embodiment AAT may be used to treat, control, or prevent autoimmune disease in patients, either with or without known AATD. A diagnosis of autoimmune disease may prompt a medical provider to test a patient for AATD. Alternatively, a patient diagnosed with autoimmune disease may seek genetic testing for AATD through an independent genetic testing company such as 23andMe or geneology.com, a pharmaceutical company supplied test kit or other private methods available.

A method for treating a patient suffering from autoimmune disease with AAT begins with the determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Bone Marrow Disease

Bone marrow is a soft fatty tissue found inside the central spongy part of bones throughout the skeleton and is the body's primary producer of white blood cells, red blood cells, and platelets. There are five different types of white blood cells: lymphocytes, neutrophils, eosinophils, basophils, and monocytes. Each type of white blood cell plays a different role in protecting the body from infection.

The chemotherapy and radiation that is typical in cancer treatment causes severe damage to bone marrow; thus, the body's normal production of white blood cells, red blood cells, and platelets is adversely affected. A method of preventing cancer is provided such that the use of chemotherapy and radiation is reduced or eliminated and damage to bone marrow is reduced or eliminated.

Neutrophils, basophils, and eosinophils kill and digest bacteria. Neutrophils migrate to the site of an infection in the body to begin killing the invading microbes. However, neutrophils release enzymes such as Neutrophil Elastase (“NE”), Cathepsin G (“CG”), Proteinase 3, and others that target and destroy thrombospondin 1 (“tsp1” or “thbs1”), a protein that acts as a natural inhibitor of neovascularization and tumorigenesis. When bacterial or virus control is not necessary, serine protease activity of neutrophils is generally not needed, and can reduce the amount of tsp1 available to do the body's natural work against cancer and performing other functions. By controlling and maintaining a balance of neutrophils with AAT, unnecessary inflammation can be reduced or eliminated, while allowing tsp1 and AAT to prevent potential cancer cells from developing, existing cancer cells from maturing, and existing tumors from metastasizing. The presence of healthy tsp1 cells can greatly diminish the spread of metastasizing cells and can help naturally fight off the development and spread of cancer. The role tsp1 can play in cancer treatment is described by Martin-Manso in their research paper describing how tsp1 interferes with tumor progression. Additionally, Chang et al demonstrated that overexpression of AAT promoted angiogenesis and cell adhesion through increasing expression of tsp1. In contrast, down-regulation of AAT by short hairpin RNA suppressed cell proliferation, metastasis, and adhesion in human lung adenocarcinoma A549 cells and in the lung tissue of K-ras LA1 lung cancer model mice. These findings strongly suggest that AAT regulation shows promise as an alternative avenue for lung cancer treatment and prevention.

Tsp1 protects against metastasis of cancer cells, especially within the lungs. Neutrophils can destroy both lung tissue in the lungs called alveoli and tsp1. This destruction creates an environment that is favorable to the spread of cancer and illness within the lungs and throughout the entire body.

Due to the nature of the interaction between AAT and tsp1, in one embodiment AAT may be used to treat, control, or prevent the destruction of tsp1 in patients, either with or without known AATD. A diagnosis of cancer or other condition associated with destruction or degradation of tsp1 may prompt a medical provider to test a patient for AATD. Alternatively, such a patient may seek genetic testing for AATD may seek genetic testing for AATD through an independent genetic testing company such as 23andMe or geneology.com, a pharmaceutical company supplied test kit or other private methods available.

A method for treating a patient suffering from bone marrow disease, cancer or other condition associated with destruction or degradation of tsp1 with AAT begins with the step of determining if the patient is AAT deficient or in the state of having low circulating AAT without being AAT deficient. Determining if the patient is AAT deficient proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from cancer, the administered AAT counters excessive NE in the patient's system, allowing tsp1 levels to increase; thus curing, controlling, or reducing the severity of the patient's cancer.

Through the use of AAT to treat cancer, the use of chemotherapy and radiation is reduced, and the patient's bone marrow is protected from damage. Treatment using AAT rather than radiation therapy or chemotherapy avoids the many side effects of radiation and chemotherapy including low blood cell counts that can result from these invasive treatments.

Cancer

When neutrophils permeate the cells of cysts, tumors, or tissue, those cells now have potential to suffer unhealthy consequences including the development of cancer. When cancer cells enter the circulatory system, there is potential for metastasizing. As noted by Anette Breindl's article, “Neutrophils are the most common type of white blood cells and, as such, act as the infantrymen of the innate immune system. One of their weapons is the formation of neutrophil extracellular traps (NETs)—sticky packages of DNA, histones and proteases that are meant to catch and digest bacterial invaders. Egeblad and her colleagues identified the role of NETs in metastasis because in previous work on metastatic breast cancer, they had “noticed that these metastatic breast cancer cells were attracting a lot of neutrophils,” she said. Other researchers had reported that, in contrast, neutrophils, under certain circumstances, appeared to promote tumor metastasis.”

AAT levels may be monitored throughout the life of a patient to determine a baseline level for circulating AAT, which may serve as a predictor of the patient's potential for developing cancer.

Breast Cancer

Many forms of breast cancer exist, including: metastatic breast cancer (“MBC”), ductal carcinoma in Situ (“DCIS”), invasive ductal carcinoma (“IDC”), triple negative breast cancer, inflammatory breast cancer (“IBC”), medullary, tubular carcinoma, mucinous carcinoma, Paget disease, small cell carcinoma of the breast, and non-small cell cancer.

Small cell carcinoma is strongly associated with smoking. Secondhand smoke is associated with approximately 30% of this type of cancer. Smoking is known to contribute to the destruction of AAT. AAT together with thrombospondin1 (“tsp1” or “thbs1”), a protein that acts as a natural inhibitor of neovascularization and tumorigenesis, maintain the health of the lungs, breast tissue, and throughout the entire human body of a patient.

With ample amounts of circulating AAT through the patient's body, the defense mechanism of tsp1 and AAT can defeat excess neutrophils' destructive forces, therefore protecting the lungs, chest/breast tissue and the entire body.

Colorectal Cancer

The use of neutrophil elastase (“NE”) as a diagnostic marker and therapeutic target in colorectal cancers is described by Ho et al.

Melanoma and Skin Cancers

Melanoma and other skin cancers may benefit from the administration of AAT, including treatment by local or topical application post-surgery. Active AAT reduces neutrophils and also helps to maintain and protect any Thrombospondin1 (“tsp1” or “thbs1”) that the body's natural defense system is sending to an area of the skin to fight off cancer.

Ovarian Cancer

AAT may be administered to control tissue infiltration of the ovaries by neutrophils and subsequent growth of tumors and cysts which could potentially form cancer cells and then metastasize.

Pancreatic Cancer

Intraductal papillary mucinous neoplasm (“IPMN”) is a type of tumor that can occur within the cells of the pancreatic duct. IPMN tumors produce mucus, and this mucus can form pancreatic cysts. Although IPMNs are benign tumors, they can progress to pancreatic cancer; therefore, IPMN is viewed as a precancerous condition. Traditional management options include close monitoring and early prophylactic resection procedure.

AAT may be administered to control tissue infiltration of the pancreas by neutrophils and subsequent growth of tumors and cysts which could potentially form cancer cells and then metastasize. AAT may be used to prevent, control, and/or shrink IPMN, cysts, and pancreatic cancer.

Prostate Cancer

AAT may be administered to control tissue infiltration of the prostate by neutrophils and subsequent growth of tumors and cysts which could potentially form cancer cells and then metastasize.

Lerman et al describe how cancer develops, and in particular show the mechanism by which neutrophil elastase (“NE”) promotes prostate cancer progression.

Testicular Cancer

AAT may be administered to control tissue infiltration of the testicles by neutrophils and subsequent growth of tumors and cysts which could potentially form cancer cells and then metastasize.

Uterine Cancer

AAT may be administered to control tissue infiltration of the uterus by neutrophils and subsequent growth of tumors and cysts which could potentially form cancer cells and then metastasize.

Blaisdell et all describe the role of neutrophils in uterine tumor cells.

Treating Cancer with AAT

Grecian et al describes the role of neutrophils in cancer. The human body is capable of producing large amounts of neutrophil elastase (“NE”) and other neutrophils. Without proper inhibition of excessive neutrophils and NE, the body can easily enter a state of excessive inflammation. When these neutrophils are not properly used or inhibited, they can transform a healthy body to an unhealthy one by many mechanisms, for example by the formation of neutrophil extracellular traps which infiltrate tumors where they can become benign or malignant tumors that can potentially metastasize and circulate throughout the body and form multiple forms of cancer.

Treffers et al describes further research concerning the role of neutrophils in cancer and their active role in the progression of cancer.

Coffelt et al provides further evidence of the role of neutrophils in cancer, stating that in patients with solid cancers, neutrophils numbers expand both in the tumor microenvironment and systematically, and are generally associated with poor prognosis. Neutrophils influence tumor initiation, growth, and metastasis. Neutrophils exert multifaceted and sometimes opposing roles during cancer initiation, growth, and dissemination. Depending on the spectrum and quantity of soluble mediators produced by cancer cells and cancer-associated cells, neutrophils can be polarized into different activation states by which they elicit various pro- or antitumor functions.

As shown by Rayes et al, inflammation in the lungs increases the risk of cancer in the lung. Pre-existing inflammation in the lungs may increase the risk that cancers beginning elsewhere will spread to the lungs.

A study by Garcia-Orad et al, suggests that AATD favors invasion by cancer cells, leaving individuals with AATD at greater risk for developing cancer.

Oxidative stress contributes to the development of cancer. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of cancer through suppression of oxidative stress and inhibition of neutrophil elastase.

Due to the link between AATD and cancer, in one embodiment AAT may be used in preventing, lessening the negative effects of, controlling, and treating cancer in patients, either with or without known AATD. A diagnosis of cancer may prompt a medical provider to test a patient for AATD or for having lower AAT levels without being AATD. Alternatively, a patient diagnosed with cancer may seek genetic testing for AATD through an independent genetic testing company such as 23andMe or geneology.com. Alternatively, a patient may self-test for AATD using an independent testing company.

A method for treating a patient suffering from cancer with AAT begins with the step of determining if the patient is AAT deficient or has low circulating AAT. Determining if the patient is AAT deficient or has low circulating AAT proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

As shown by Stockley, AAT has been recognized as a potent inhibitor of serine proteinases. Through the administration of AAT to a patient suffering from cancer, the formation or metastasis of cells containing neutrophil elastase (“NE”), cathepsin G, proteinase 3, and other serine proteases is prevented. Over time, if these serine proteases are allowed to attach to and permeate cells, the neutrophils may solidify, form neutrophil extracellular traps, and allow for the presence and ability to develop abnormal cells with malignant potential, allowing many diseases, including Alzheimer's disease, ALS, sickle cell anemia, and others, to create harm and spread throughout the body. In other terms, one condition is causing another condition to manifest elsewhere in the body, this occurrence is called a comorbidity. For example, AATD patients have a liver disease that often causes emphysema and genetic COPD.

In addition, through the administration of AAT to a patient suffering from cancer, the formation or metastasis of benign and malignant cancer cells is prevented. These cells can contain or develop abnormal cells with malignant cancer potential, and AAT prevents these cells from being circulated throughout the body and inhibits them from metastasizing elsewhere in the body.

By preventing and treating cancer with AAT, the use of chemotherapy and radiation is reduced, reducing the pain, psychological damage, expense, and other societal consequences associated with the disease. Side effects of chemotherapy and radiation include hair loss, fatigue, nausea, vomiting, diarrhea, constipation, infection, weakness, bleeding, pain, “Chemo brain,” skin issues and problems, breast problems, rib fractures, lymphedema, heart problems, nerve problems, bone marrow issues, immunosuppression of the immune system, and low blood cell counts, all of which can be reduced through the treatment of cancer using AAT.

Circulation Problems

Raynaud's disease causes some areas of the body, often fingers or toes, to feel numb and cold in response to cold temperatures or stress. In Raynaud's disease, smaller arteries that supply blood to the skin narrow, limiting blood circulation to affected areas.

Due to the link between AATD and Raynaud's disease and other circulation problems, in one embodiment AAT may be used to treat, control, or prevent circulation problems in patients, either with or without known AATD. A diagnosis of circulation problems may prompt a medical provider to test a patient for AATD. Alternatively, a patient diagnosed with circulation problems may seek genetic testing for AATD through an independent genetic testing company such as 23andMe or geneology.com, a pharmaceutical company supplied test kit or other private methods available.

A method for treating a patient suffering from circulation problems with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from circulation problems, the administered AAT cures, controls, or reduces the severity of the patient's circulation problems.

Coronavirus

The American Thoracic Society has published that Neutrophil Elastase (“NE”) can cause the neutralization of other neutrophils' ability to fight off bacteria. As documented by McCarthy et al, AAT inhibits neutrophil elastase (“NE”) in the lungs. AAT also inhibits other neutrophil-derived proteases. Thus, AAT protects lung tissue from degradation. There is a significantly higher burden of neutrophils in the lungs of individuals with AATD compared with healthy individuals, and the significant neutrophil burden contributes to increased proteolytic activity and inflammation. Further, NE impairs the ability of other neutrophils to kill bacteria within the lungs.

A cytokine storm occurs when there is an excessive and uncontrolled release of pro-inflammatory cytokines. It can result in acute respiratory distress syndrome (“ARDS”), multiple organ failure, and death. Cytokine storms have been seen in severe SARS, MERS, and COVID-19 cases, and pneumonia is a frequent complication of these conditions. See Mangalmurti. Clinical reports from China published by Zhang et al. in the journal Clinical Immunology (Zhang et al 2020), revealed that, in addition to lymphocytopenia, COVID-19 patients had high inflammatory parameters and proinflammatory cytokines, particularly IL2, IL6, IL7, IL8, IL10, and TNFa. Inflammatory cytokines were further elevated in patients admitted to the ICU7, all suggestive of a COVID-19 induced cytokine storm. Autopsy reports from COVID-19 patients found atrophy in the spleen and secondary lymphoid tissues. Since these organs do not express ACE2, the receptor for COVID-19 infection, Zhang et al. theorize that damage to the immune system was a result of a cytokine storm. Zhang et al. conclude, in agreement with Dr. Roth, that in light of the real possibility of a cytokine storm in critically ill patients, an anti-inflammatory treatment may be necessary, but that “a timely anti-inflammation treatment initiated at the right window of time is of pivotal importance and should be tailored in individual patients to achieve the most favorable effects.” Treatment of cytokine storms by timely administration of an anti-inflammatory agent is further discussed in Kimberly B. Bjugstad's article.

Due to the link between AATD and coronavirus, in one embodiment AAT may be used to treat, control, or prevent coronavirus in patients, either with or without known AATD. A diagnosis of coronavirus may prompt a medical provider to test a patient for AATD. Alternatively, a patient diagnosed with coronavirus may seek genetic testing for AATD through an independent genetic testing company such as 23andMe or geneology.com, a pharmaceutical company supplied test kit or other private methods available.

A method for treating a patient suffering from coronavirus with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from coronavirus, the administered AAT inhibits NE that contributes to coronavirus; thus curing, controlling, or reducing the severity of the patient's coronavirus. In the case of treating a coronavirus, subsequent coronavirus, ARDS, pneumonia and/or cytokine storms, medical providers may administer AAT more frequently as neutrophil-lymphocyte count ratios (NLCR) can be monitored in these emergency situations by medical providers hour by hour. By using AAT to control NE, coronavirus may be controlled or prevented before it ever develops; therefore, reducing or eliminating the need to use antibiotics to treat coronavirus and preventing bacteria from becoming antibiotic resistant.

Cushing's Disease

Pituitary pars intermedia dysfunction (“PPID”), also known as Cushing's disease, Cushing's syndrome, or hypercortisolism, is a hormonal disorder that occurs when the body is exposed to excess levels of the hormone cortisol for an extended period of time. Signs of Cushing's disease include a fatty hump between the shoulders, a rounded face, and pink or purple stretch marks on the skin. Cushing's disease can also result in high blood pressure, bone loss, and type 2 diabetes.

Cushing's disease affects humans and horses. The effect of Cushing's disease on immunity in horses was shown by McFarlane et al.

Due to the link between AATD and Cushing's disease, in one embodiment AAT may be used to treat, control, or prevent Cushing's disease in patients, either with or without known AATD. A diagnosis of Cushing's disease may prompt a medical provider to test a patient for AATD. Alternatively, a patient diagnosed with Cushing's disease may seek genetic testing for AATD through an independent genetic testing company such as 23andMe or geneology.com, a pharmaceutical company supplied test kit, or other private methods available.

A method for treating a patient suffering from Cushing's disease with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from Cushing's disease, the administered AAT cures, controls, or reduces the severity of the patient's Cushing's disease.

Cystic Fibrosis

Due to the link between AATD and cystic fibrosis, in one embodiment AAT may be used to treat and control cystic fibrosis in patients, including the side effects of cystic fibrosis, either with or without known AATD. A diagnosis of cystic fibrosis may prompt a medical provider to test a patient for AATD. Alternatively, a patient diagnosed with cystic fibrosis may seek genetic testing for AATD through an independent genetic testing company such as 23andMe or geneology.com, a pharmaceutical company supplied test kit or other private methods available.

A method for treating a patient suffering from cystic fibrosis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from cystic fibrosis, the administered AAT may control, delay the onset of, or reduce the severity of comorbid conditions associated with the patient's cystic fibrosis.

Demyelinating Diseases

A demyelinating disease is any condition that results in damage to myelin sheath, which is the protective covering that surrounds nerve fibers in the brain, optic nerves, and spinal cord. When the myelin sheath is damaged, nerve impulses slow or even stop, causing neurological problems. Demyelinating diseases include multiple sclerosis, optic neuritis, neuromyelitis optica, transverse myelitis, adrenoleukodystrophy, adrenomyeloneuropathy, and other conditions. Demyelination is a factor in dementia and Alzheimer's disease. The blood brain barrier can be infiltrated when NE is uninhibited. Nerve fibers can suffer degradation from excess neutrophil elastase damaging nerve cells.

Multiple sclerosis (“MS”) is the most common demyelinating disease of the central nervous system. MS is an unpredictable disease of the central nervous system that disrupts the flow of information within the brain, and between the brain and body. MS involves an immune-mediated process in which an abnormal response of the body's immune system is directed against the central nervous system. Within the central nervous system, the immune system causes inflammation that damages myelin, which is the fatty substance that surrounds and insulates the nerve fibers, as well as the nerve fibers themselves and the specialized cells that make myelin. When myelin or nerve fibers are damaged or destroyed in MS, messages within the central nervous system are altered or stopped completely. Damage to areas of the central nervous system may produce a variety of neurological symptoms that will vary among people with MS in type and severity. The damaged areas develop scar tissue which gives the disease its name. The cause of MS is not known, but it is believed to involve genetic susceptibility, abnormalities in the immune system, and environmental factors that combine to trigger the disease.

The cerebral microcapillary endothelium forms a highly important barrier between the blood and the interstitial fluid of the brain (blood-brain barrier) that controls the passage of molecules and cells in and out of the central nervous system. A study by von Wedel-Parlow et al demonstrates that neutrophils cross the blood brain barrier primarily on transcellular pathways. As shown by Joice et al, resting neutrophils induce acute reductions in permeability of the blood brain barrier. Moxon-Emre and Schlichter demonstrated that neutrophil depletion reduces breakdown of the blood brain barrier, axon injury, and inflammation after intracerebral hemorrhage. Sayed et al found that neutrophils promote breach of the blood-brain barrier and together with T cells lead to further inflammatory cell influx and myelin damage, findings that provide specific targets for intervention in multiple sclerosis as well as other immune-mediated central nervous system diseases. Aubé et al showed further support for neutrophils' role in blood brain barrier disruption, finding that neutrophils are involved in the initial events that take place during EAE and that they are intimately linked with the status of the blood brain barrier and blood spinal cord barrier, disruption of which are hallmarks of MS, its animal model experimental autoimmune encephalomyelitis (“EAE”), and neuromyelitis optica (“NMO”).

As a powerful inhibitor of inflammation, in one embodiment AAT may be used to treat, control, or prevent MS in patients, either with or without known AATD. A diagnosis of MS may prompt a medical provider to test a patient for AATD. Alternatively, a patient diagnosed with MS may seek genetic testing for AATD through an independent genetic testing company such as 23andMe or geneology.com, a pharmaceutical company supplied test kit or other private methods available.

A method for treating a patient suffering from MS with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from MS, the myelin sheath cells throughout the entire body can be protected and inflammation is reduced; thus, the patient's MS may be cured, controlled, or reduced in severity, and damage to the nerve myelin sheath may be prevented.

Depression

Depression is a mood disorder that causes a persistent feeling of sadness and loss of interest. Also called major depressive disorder or clinical depression, it affects how a patient feels, thinks, and behaves, and can lead to a variety of emotional and physical problems.

Oxidative stress contributes to the development of depression. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of depression through suppression of oxidative stress.

Due to the link between AATD and depression, in one embodiment AAT may be used to treat, control, or prevent depression in patients, either with or without known AATD. A diagnosis of depression may prompt a medical provider to test a patient for AATD. Alternatively, a patient diagnosed with depression may seek genetic testing for AATD through an independent genetic testing company such as 23andMe or geneology.com, a pharmaceutical company supplied test kit, or other private methods available.

A method for treating a patient suffering from depression with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from depression, the patient's condition may be cured, controlled, or reduced in severity. Depression symptoms may be further relieved when pain comorbidities, and other factors of poor health that can influence depression are effectively assisted by second hand positive effects of AAT therapy as it circulates throughout the entire body.

Diabetes

Due to the link between AATD and diabetes, in one embodiment AAT may be used in preventing, lessening the negative effects of, and treating diabetes in patients, either with or without known AATD. A diagnosis of diabetes may prompt a medical provider to test a patient for AATD or lower AAT levels without being AATD. Alternatively, a patient diagnosed with diabetes may seek genetic testing for AATD through an independent genetic testing company such as 23andMe or geneology.com, a pharmaceutical company supplied test kit or other private methods available.

A method for treating a patient suffering from diabetes with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from diabetes, the patient's condition may be cured, controlled, or reduced in severity.

Ears and Hearing

Administration of AAT may control formation of neutrophil extracellular traps within the ears, including excessive neutrophils within the ear canals, especially in babies and young children. With AAT, children's earaches and the use of tubes can be prevented by measuring excess neutrophil counts in babies and toddlers, saving procedures and suffering. Administration of AAT may also prevent inner ear pain and hearing loss associated with otosclerosis, otospongiosis, and other conditions affecting the ears and hearing.

Excessive neutrophil elastase (“NE”) within the ear can result in excessive fluid in the ear. AAT may be used to eliminate inflammatory response in the inner ear. The link between NE and chronic otitis has been demonstrated by Tierney et al.

The role of excess neutrophils in inner ear infections, bacterial meningitis, and other conditions was shown by Hamano et al.

Due to the link between AATD and conditions affecting the ears, and/or hearing, in one embodiment AAT may be used to treat, control, or prevent conditions affecting the ears, and hearing in patients, either with or without known AATD. A diagnosis of a condition affecting the ears, and/or hearing may prompt a medical provider to test a patient for AATD. Alternatively, a patient diagnosed with a condition affecting the ears and hearing may seek genetic testing for AATD through an independent genetic testing company such as 23andMe or geneology.com, a pharmaceutical company supplied test kit or other private methods available.

A method for treating a patient suffering from a condition affecting the ears, and/or hearing with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended. AAT may be administered in the form of a powder, tablet, or liquid, or AAT may be inhaled, infused, or taken in another manner. Currently Kamada is the producer of the product Glassia. Glassia has been in trial to treat patients with AAT in the form of an aerosol applied with a nebulizer at a current rate of 80 mg twice daily for a total of 160 mg per day. Kamada received FDA approval in 2019. Using this dosage as an adult standard, the physician must take into consideration the 60 mg/kg of weight standard of typical AAT infusion therapy. With direct application into the inner and middle ear, splitting the dosage into multiple application dosages throughout the day over a 24-hour period and the desired number of treatments per day. The described dosages of 160 mg per day is recommended for adults, and this dosage is not appropriate for infants and babies. For infants and babies, direct application of the AAT mist is applied directly into the ears to control excess neutrophils within the middle and inner ear.

Through the administration of AAT to a patient suffering from a condition affecting the ears, and/or hearing, the patient's condition may be cured, controlled, or reduced in severity.

Eclampsia, Preeclampsia, and Hypertension

Eclampsia and preeclampsia are characterized by high blood pressure in pregnant women, possibly leading to convulsions or stroke. Acute kidney and liver failure can occur in the mother, and the infant can suffer severe hypoxia. A link between neutrophil extracellular traps (“NETs”) and several conditions including preeclampsia was shown by Brinkmann and Zychlinsky. Further connections between neutrophils and preeclampsia were shown by Leik and Walsh, by Canzoneri et al, and by Clark et al. Preeclampsia and its complications have become a leading cause of maternal and fetal morbidity and mortality. The development of preeclampsia is unpredictable; therefore, it is difficult to prevent and manage, yet a monitoring of circulating alpha1 antitrypsin throughout pregnancy can make it much more predictable and dealt with appropriately.

AAT can relieve medical situations that can cause high blood pressure or hypertension by reducing NETs that can clog blood vessels. By the elimination of NETs, the circulatory system can be maintained in a healthier state. AAT attaches to the excess neutrophils that cause NETs to form, and AAT assists with the safe elimination of NETs from the body.

Oxidative stress contributes to the development of preeclampsia and other forms of hypertension. Taggart et al describe in detail the role of oxidative stress on the development of preeclampsia. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of preeclampsia through suppression of oxidative stress.

In addition, El-Eshmawy et al have linked neutrophil elastase (“NE”) to prehypertension and airflow limitation in obese women. AAT may also prevent the development of preeclampsia through inhibition of NE.

Due to the link between AATD and eclampsia, preeclampsia, and hypertension, in one embodiment AAT may be used to treat, control, or prevent eclampsia, preeclampsia, and hypertension in patients, either with or without known AATD. A diagnosis of eclampsia or preeclampsia or hypertension may prompt a medical provider to test a patient for AATD. Alternatively, a patient diagnosed with eclampsia or preeclampsia, or hypertension may seek genetic testing for AATD through an independent genetic testing company such as 23andMe or geneology.com, a pharmaceutical company supplied test kit or other private methods available.

A method for treating a patient suffering from eclampsia or preeclampsia or hypertension with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previously AATD patients have commonly only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from eclampsia or preeclampsia or hypertension, the patient's condition may be cured, controlled, or reduced in severity. By reducing the effects of eclampsia and preeclampsia, maternal morbidity and mortality and adverse neonatal outcomes will be reduced, and the cost burden to the U.S. health care system demonstrated by Stevens et al will also be reduced.

Endometriosis

Excessive neutrophil elastase (“NE”) is associated with endometriosis. Endometriosis is characterized by the implantation and growth of endometriotic tissues outside the uterus. A study by Izumi et al showed that peritoneal neutrophils and macrophages secrete biochemical factors that help endometriotic cell growth and invasion, and angiogenesis, suggesting a role between neutrophils and endometriosis. Further, a study by Mueller et al showed that neutrophils infiltrating the endometrium express vascular endothelial growth factor and have a potential role in endometrial angiogenesis. AAT is a powerful inhibitor of neutrophils; therefore, AAT may be used to treat and prevent endometriosis and related conditions.

As a powerful inhibitor of NE, in one embodiment AAT may be used to treat, control, or prevent endometriosis in patients, either with or without known AATD. A diagnosis of endometriosis may prompt a medical provider to test a patient for AATD. Alternatively, a patient diagnosed with endometriosis may seek genetic testing for AATD through an independent genetic testing company such as 23andMe or geneology.com, a pharmaceutical company supplied test kit or other private methods available.

A method for treating a patient suffering from endometriosis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended. AAT may be administered in the form of a powder, tablet, or liquid, or AAT may be inhaled, infused, or taken in another manner. AAT dosage should be calculated as a function of the current level of circulating AAT in the patient plus the additional amount of AAT required to place the patient within the standard range of MN allele individuals. This range typically is described as being between 100-300 mg/dL. Previous AATD patients have only been brought up to the lower end of this range. Dosages should be made available every 6 to 7 days as the life of AAT, according to Prolastin-C maker Grifols, is 156 hours, which is approximately 6.5 days. Typical augmentation therapy dosage for AATD patients is 60 mg/kg of weight, but physicians have discretion to prescribe AAT to a patient based on current circulating levels of AAT. By correlating the typical dosage to circulating AAT levels currently in individuals, this becomes an alternative method to mathematically calculate dosages of AAT for patients with the conditions described within this disclosure. Currently Kamada is the producer of the product Glassia. Glassia has been in trial to treat patients with AAT in the form of an aerosol applied with a nebulizer at a current rate of 80 mg twice daily for a total of 160 mg per day. Kamada received FDA approval in 2019. Applications for endometriosis may require multiple direct injections to the areas affected by endometriosis, applicated by the dosage standard of 60 mg/kg of weight, divided by the frequency and time frame direct injection treatment continues, while at the same time coordination of other types of AAT therapy that may be suggested by the physician to keep the overall levels of circulating AAT in the patient's body in a suggested minimum range of over 120 mg/dL.

Through the administration of AAT to a patient suffering from endometriosis, the patient's endometriosis and related conditions including pelvic abscess, neutrophils' infiltration of the endometrium expressing vascular endothelial growth factor (a potential contributor to endometrial angiogenesis), infertility, endometriosis-related irritable bowel syndrome, pathophysiology of endometriosis, endometrial angiogenesis, tumor growth, cancer cell development and growth, and cancer cell metastasizing are cured, controlled, or reduced in severity.

Epilepsy

Epilepsy is a neurological disorder marked by sudden recurrent episodes of sensory disturbance, loss of consciousness, or convulsions. Epilepsy is associated with abnormal electrical activity in the brain. As demonstrated by an eHealthMe study, neutrophil count is increased in patients with epilepsy. By reducing neutrophil count using AAT, epilepsy and epileptic seizures may be managed.

Due to the link between AATD and epilepsy, in one embodiment AAT may be used to treat, control, or prevent epilepsy in patients, either with or without known AATD. A diagnosis of epilepsy may prompt a medical provider to test a patient for AATD. Alternatively, a patient diagnosed with epilepsy may seek genetic testing for AATD through an independent genetic testing company such as 23andMe or geneology.com, a pharmaceutical company supplied test kit or other private methods available.

A method for treating a patient suffering from epilepsy with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from epilepsy, the administered AAT decreases neutrophil count, thus curing, controlling, or reducing the severity of the patient's epilepsy and reducing the magnitude of epileptic seizures.

Esophagus and GERD

Gastroesophageal reflux disease (“GERD”) is a long-term condition where acid from the stomach comes up into the esophagus. Many people occasionally experience gastroesophageal reflux; however, if an individual experiences acid reflux more than twice a week, they may be diagnosed with GERD. Patients suffering from conditions affecting the esophagus, including acid indigestion, GERD, and esophageal carcinoma, are actually suffering manifestations caused by excess neutrophil elastase (“NE”). Coughing caused by varying meal types can be a sign of inflammation that should be monitored and tracked as proper maintenance of the alimentary canal. AAT is a powerful inhibitor of NE.

Due to the link between AATD and conditions affecting the esophagus, in one embodiment AAT may be used to treat, control, or prevent conditions affecting the esophagus in patients, either with or without known AATD. A diagnosis of a condition affecting the esophagus may prompt a medical provider to test a patient for AATD. Alternatively, a patient diagnosed with a condition affecting the esophagus may seek genetic testing for AATD through an independent genetic testing company such as 23andMe or geneology.com, a pharmaceutical company supplied test kit or other private methods available.

A method for treating a patient suffering from a condition affecting the esophagus with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from a condition affecting the esophagus, the administered AAT decreases NE, thus curing, controlling, or reducing the severity of the patient's condition.

Eyes

Patients suffering from conditions affecting the eyes, including retinal problems, macular degeneration, floaters, and angiogenesis, are suffering manifestations caused by excess neutrophil elastase (“NE”). AAT is a powerful inhibitor of NE.

Due to the link between AATD and conditions affecting the eyes, in one embodiment AAT may be used to treat, control, or prevent conditions affecting the eyes in patients, either with or without known AATD. A diagnosis of a condition affecting the eyes may prompt a medical provider to test a patient for AATD. Alternatively, a patient diagnosed with a condition affecting the eyes may seek genetic testing for AATD through an independent genetic testing company such as 23andMe or geneology.com, a pharmaceutical company supplied test kit or other private methods available.

A method for treating a patient suffering from a condition affecting the eyes with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from a condition affecting the eyes, the administered AAT decreases NE, thus curing, controlling, or reducing the severity of the patient's condition.

Fibrosis

Fibrosis is characterized by the overgrowth, hardening, and/or scarring of various tissues. Fibrosis is attributed to excess deposition of extracellular matrix components including collagen. Fibrosis is the end result of chronic inflammatory reactions induced by a variety of stimuli including persistent infections, autoimmune reactions, allergic responses, chemical insults, radiation, and tissue injury. AAT is a powerful inhibitor of inflammation.

Due to the link between AATD and fibrosis, in one embodiment AAT may be used to treat, control, or prevent fibrosis in patients, either with or without known AATD. A diagnosis of fibrosis may prompt a medical provider to test a patient for AATD. Alternatively, a patient diagnosed with fibrosis may seek genetic testing for AATD through an independent genetic testing company such as 23andMe or geneology.com, a pharmaceutical company supplied test kit or other private methods available.

A method for treating a patient suffering from fibrosis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from fibrosis, inflammation is reduced; thus curing, controlling, or reducing the severity of the patient's fibrosis.

Heart Disease

Oxidative stress contributes to the development of heart disease. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g., O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of heart disease through suppression of oxidative stress.

Following an acute heart attack, an immune response occurs. This immune response releases massive amounts of immune cells known as neutrophils to the heart and surrounding area, causing inflammation that damages the muscles of the heart and the area surrounding the heart. Well-timed applications of AAT can protect the body for the long term negative and destructive effects that heart attacks can and do have on a patient's heart. Administration of AAT when circulating levels fall below the range of 100-300 mg/dL can prevent further damage to a patient due to heart disease, cardiovascular disease, and disease of the circulatory system. Furthermore, by preventing inflammation, administration of AAT can potentially prevent future heart attacks.

Due to the link between AATD and heart disease, in one embodiment AAT may be used to treat, control, or prevent heart disease in patients, either with or without known AATD. A diagnosis of heart disease may prompt a medical provider to test a patient for AATD. Alternatively, a patient diagnosed with heart disease may seek genetic testing for AATD through an independent genetic testing company such as 23andMe or geneology.com, a pharmaceutical company supplied test kit or other private methods available.

A method for treating a patient suffering from heart disease with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from heart disease, inflammation is reduced; thus curing, controlling, or reducing the severity of the patient's heart disease and potentially preventing or reducing the severity of a heart attack. In the case of treating a heart attack patient, medical providers may administer AAT more frequently as neutrophil-lymphocyte count ratios (NLCR) can be monitored often.

Through the administration of AAT to a patient who is having, or has recently suffered from a heart attack, the administered AAT inhibits neutrophils and/or NE that contribute to inflammation; thus curing, controlling, or reducing the severity of the patient's post heart attack situation. By using AAT to control neutrophils, and/or NE, the post heart attack condition may be controlled and/or prevent further heart damage and further complications.

Hemophilia

Adult-onset hemophilia and acquired hemophilia are rare autoimmune disorders characterized by bleeding that occurs in patients with a personal and family history negative for hemorrhages. It occurs when the immune system produces antibodies that mistakenly attack healthy tissue, specifically specialized proteins known as clotting factors. Affected individuals experience abnormal, uncontrolled bleeding into the muscles, skin, and soft tissue during surgery or following trauma.

Thrombin is a protein for clotting. Low levels of AAT increase antithrombin. Without thrombin to convert fibrinogen to fibrin, the expression of tsp1 is also reduced. Tsp1 is needed to bind with fibrin and activate platelets.

Due to the link between AATD and hemophilia, in one embodiment AAT may be used to treat, control, or prevent hemophilia or improve platelet count in patients, either with or without known AATD. A diagnosis of hemophilia may prompt a medical provider to test a patient for AATD. Alternatively, a patient diagnosed with hemophilia may seek genetic testing for AATD through an independent genetic testing company such as 23andMe or geneology.com, a pharmaceutical company supplied test kit or other private methods available.

A method for treating a patient suffering from hemophilia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from hemophilia, the administered AAT increases levels of tsp1 needed for normal clotting; thus curing, controlling, or reducing the severity of the patient's hemophilia and/or improving the patient's platelet count.

Infertility and Sterility

As shown by Mueller et al, neutrophils infiltrating the endometrium express vascular endothelial growth factor, potentially have a role in endometrial angiogenesis. This potential role in endometrial angiogenesis is also indicative of a role of neutrophils in female fertility and sterility.

As a powerful inhibitor of neutrophils, in one embodiment AAT may be used to treat, control, or prevent female infertility and sterility, either in patients with or without known AATD. A diagnosis of infertility or sterility may prompt a medical provider to test a patient for AATD. Alternatively, a patient diagnosed with infertility or sterility may seek genetic testing for AATD through an independent genetic testing company such as 23andMe or geneology.com, a pharmaceutical company supplied test kit or other private methods available.

A method for treating a patient suffering from infertility or sterility with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from infertility or sterility, the administered AAT decreases neutrophil levels; thus curing, controlling, or reducing the severity of the patient's infertility or sterility.

Inflammation and Chronic Inflammation

Inflammation is a vital part of the immune system's response to injury and infection. Inflammation signals the immune system to heal and repair damaged tissue and defend the body against foreign invaders, such as viruses and bacteria. Without inflammation as a physiological response, wounds would fester, and infections could become deadly. However, if the inflammatory process goes on for too long or if the inflammatory response occurs in places where it is not needed, it can become problematic. Chronic inflammation has been linked to certain diseases such as heart disease or stroke, and may also lead to autoimmune disorders, such as rheumatoid arthritis and lupus.

As noted by Blaisdell et al, inflammation pervades virtually all forms of cancer even from the earliest stages of tumor development. Danger signals emanating from the tumor elicit local production of inflammatory cytokines and chemokines that subsequently draw inflammatory leukocytes into the neoplastic tissue. In general, inflammation is thought to nourish tumor growth and accelerate malignant progression.

As noted by McDaniel et al, pathogenesis of chronic venous leg ulcers is related to the prolonged presence of high numbers of activated neutrophils secreting proteases in the wound bed that destroy growth factors, receptors, and the extracellular matrix that are essential for healing, and these factors are believed to contribute to a chronically inflamed wound that fails to heal. Similarly, Ai et al suggest that pressure ulcers, which are chronic wounds that do not heal in a timely fashion, are affected by neutrophil elastase, and that pressure ulcer fluid contains a high level of neutrophil elastase which may be involved in the delay of healing of pressure ulcers through fibronectin degradation. As noted by Kvietys and Carter, studies have implicated neutrophils in the gastric mucosal injury induced by luminal ethanol.

As noted by Liu et al 1998, activated neutrophils play an important role in tissue injury by releasing various inflammatory mediators capable of damaging endothelial cells, and their observations suggest that neutrophil elastase promotes stress-induced gastric mucosal injury by reducing gastric mucosal blood flow and increasing neutrophil accumulation.

As a powerful inhibitor of inflammation and neutrophils, in one embodiment AAT may be used to treat, control, or prevent inflammation in patients, either with or without known AATD. A diagnosis of inflammation may prompt a medical provider to test a patient for AATD. Alternatively, a patient diagnosed with inflammation may seek genetic testing for AATD through an independent genetic testing company such as 23andMe or geneology.com, a pharmaceutical company supplied test kit or other private methods available.

A method for treating a patient suffering from inflammation with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days.

Physicians may fluctuate on 5 to 8 days between treatments, or every 6 to 7 days for typical treatments when dosages are recommended.

Through the administration of AAT to a patient suffering from inflammation, the patient's inflammation is reduced, and a number of conditions may be cured, controlled, or reduced in severity.

Kidney Disease

Kidney disease can lead to the loss of renal function over time, later leading to end stage renal disease, the final stage of chronic kidney disease. Known causes of kidney disease include congenital defects of the kidney, cystic kidney disease, cystic disease, diabetes, glomerular disease, hypertension, tubule interstitial disease, unrecovered acute kidney injury, urinary tract obstruction and/or dysfunction, recurrent kidney stone disease, and vascular disease. Many of these issues can be traced to excess neutrophils and neutrophil elastase (“NE”) in the body. End-stage renal disease and chronic kidney disease are characterized by chronic inflammation and oxidative stress. As demonstrated by Bronze-da-Rocha and Santos Silva, NE inhibitors such as AAT may play a role in predicting or preventing inflammation.

Kidney stones are hard, crystalline mineral materials formed within the kidneys or the urinary tract. Kidney stones form when there is a decrease in urine volume and/or an excess of stone-forming substances in the urine. Many kidney stones pass on their own over time; however, pain medications, medications to help with the passage of urine, and sometimes surgical procedures are needed for dealing with kidney stones.

Kidney disease and kidney stones may be treated effectively with AAT. As a powerful inhibitor of inflammation, in one embodiment AAT may be used to treat, control, or prevent kidney disease in patients, either with or without known AATD. A diagnosis of kidney disease may prompt a medical provider to test a patient for AATD. Alternatively, a patient diagnosed with kidney disease may seek genetic testing for AATD through an independent genetic testing company such as 23andMe or geneology.com, a pharmaceutical company supplied test kit or other private methods available.

A method for treating a patient suffering from kidney disease with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from kidney disease, neutrophil levels and inflammation are reduced; thus, the patient's kidney disease may be cured, controlled, or reduced in severity.

Maternal Hypoxia

Hypoxia refers to insufficient oxygenation and is a prominent feature of inflammation associated with numerous disease states. Hypoxia is common in inflamed and infected tissues, which are characterized by oxygen and nutrient deprivation. Sadiku and Walmsley provide a review of literature on innate immunometabolism and discuss the role of hypoxia in innate cell metabolic reprogramming and how this determines immune responses. Specifically, hypoxia prolongs neutrophil survival.

Maternal hypoxia can lead to fetal low brain oxygenation. A fetus can suffer severe problems from maternal hypoxia, including cerebral palsy, periventricular leukomalacia, and brain damage. As described by Golan et al, magnesium sulfate has been used to reduce hypoxia-induced motor disabilities in offspring.

As shown by Coleman and Rund, asthma and epilepsy are non-obstetric conditions that may result in maternal hypoxia. Administration of AAT can prevent or control maternal hypoxia during pregnancy by preventing non-obstetric conditions that lead to hypoxia.

Due to the link between AATD and maternal hypoxia, in one embodiment AAT may be used to treat, control, or prevent maternal hypoxia in patients, either with or without known AATD. A diagnosis of maternal hypoxia may prompt a medical provider to test a patient for AATD. Alternatively, a patient diagnosed with maternal hypoxia may seek genetic testing for AATD through an independent genetic testing company such as 23andMe or geneology.com, a pharmaceutical company supplied test kit or other private methods available.

A method for treating a patient suffering from maternal hypoxia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days.

Physicians may fluctuate on 5 to 8 days between treatments, or every 6 to 7 days for typical treatments when dosages are recommended.

Through the administration of AAT to a patient suffering from maternal hypoxia, non-obstetric conditions including asthma and epilepsy are controlled, resulting in better outcomes for the mother and child.

Muscular Dystrophy

Muscular dystrophy (“MD”) is an inherited genetic condition that gradually causes a weakening of muscles. MD usually affects boys in early childhood and leads to progressively worsening disability and premature death.

Arecco et al found that neutrophil elastase (“NE”) may be a key contributor to MD. NE breaks down several proteins found in the connective tissue that is present in various organs, including muscle.

As a powerful inhibitor of NE, in one embodiment AAT may be used to treat, control, or prevent MD in patients, either with or without known AATD. A diagnosis of MD may prompt a medical provider to test a patient for AATD. Alternatively, a patient diagnosed with MD may seek genetic testing for AATD through an independent genetic testing company such as 23andMe or geneology.com, a pharmaceutical company supplied test kit or other private methods available.

A method for treating a patient suffering from MD with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from MD, NE levels and inflammation are reduced; thus, the patient's MD may be cured, controlled, or reduced in severity.

Neointimal Lesions

Neointimal lesions are vascular abnormalities that cause narrowing and even obliteration of the vessel lumen and contribute to the progressive increase in pulmonary vascular resistance that can lead to ventricular failure. As shown by Kim et al, lesions are associated with heightened lung neutrophil elastase (“NE”) activity and PA elastin degradation.

As a powerful inhibitor of NE, in one embodiment AAT may be used to treat, control, or prevent neointimal lesions in patients, either with or without known AATD. A diagnosis of neointimal lesions may prompt a medical provider to test a patient for AATD and to discover patients ongoing circulating ATT levels. Alternatively, a patient diagnosed with neointimal lesions may seek genetic testing for AATD through an independent genetic testing company such as 23andMe or geneology.com, a pharmaceutical company supplied test kit or other private methods available.

A method for treating a patient suffering from neointimal lesions with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from neointimal lesions, NE levels and inflammation are reduced; thus, the patient's neointimal lesions may be cured, controlled, or reduced in severity.

Periodontal Disease

Excessive neutrophil levels play a role in gum disease and other dental diseases. As stated by Scott and Krauss, “neutrophils are considered the key protective cell type in periodontal tissues. Histopathology of periodontal lesions indicates that neutrophils form a ‘wall’ between the junctional epithelium and the pathogen-rich dental plaque which functions as a robust antimicrobial secretory structure and as a unified phagocytic apparatus. However, neutrophil protection is not without cost and is always considered a two-edged sword in that overactivity of neutrophils can cause tissue damage and prolong the extent and severity of inflammatory periodontal diseases.” In addition to linking inflammation due to excessive neutrophils to gingivitis, Scott and Krauss provide a review of the innate and inflammatory functions of neutrophils, describe the importance and utility of neutrophils to the host response and the integrity of the periodontium in health and disease.

Excessive neutrophil levels have also been linked to recurrent oral ulcers by Liu et al and to chronic or aggressive periodontitis by Nizam et al.

A link between neutrophil extracellular traps (“NETS”) and several conditions, including periodontitis, was shown by Brinkmann and Zychlinsky.

As a powerful inhibitor of neutrophils, NETs, and inflammation, in one embodiment AAT may be used to treat, control, or prevent periodontal disease in patients, either with or without known AATD. A diagnosis of periodontal disease may prompt a medical provider to test a patient for AATD or lower AAT levels without being AATD. Alternatively, a patient diagnosed with periodontal disease may seek genetic testing for AATD through an independent genetic testing company such as 23andMe or geneology.com, a pharmaceutical company supplied test kit or other private methods available.

A method for treating a patient suffering from periodontal disease with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from periodontal disease, inflammation is reduced; thus, the patient's periodontal disease may be cured, controlled, or reduced in severity.

Pneumonia

The American Thoracic Society has published that Neutrophil Elastase (“NE”) can cause the neutralization of other neutrophils' ability to fight off bacteria. As documented by McCarthy et al, AAT inhibits neutrophil elastase (“NE”) in the lungs. AAT also inhibits other neutrophil-derived proteases. Thus, AAT protects lung tissue from degradation. There is a significantly higher burden of neutrophils in the lungs of individuals with AATD compared with healthy individuals, and the significant neutrophil burden contributes to increased proteolytic activity and inflammation. Further, NE impairs the ability of other neutrophils to kill bacteria within the lungs.

Matsuse et al demonstrated that NE plays a critical role in severe pneumonia. Levels of NE in plasma were found to be significantly higher in severe pneumonia as compared to moderate pneumonia.

NE and its relationship to cytokine storms also plays a key role in pneumonia. A cytokine storm occurs when there is an excessive and uncontrolled release of pro-inflammatory cytokines. It can result in acute respiratory distress syndrome (“ARDS”), multiple organ failure, and death. Cytokine storms have been seen in severe SARS, MERS, and COVID-19 cases, and pneumonia is a frequent complication of these conditions. See Mangalmurti. Clinical reports from China published by Zhang et al. in the journal Clinical Immunology (Zhang et al 2020), revealed that, in addition to lymphocytopenia, COVID-19 patients had high inflammatory parameters and proinflammatory cytokines, particularly IL2, IL6, IL7, IL8, IL10, and TNFa. Inflammatory cytokines were further elevated in patients admitted to the ICU7, all suggestive of a COVID-19 induced cytokine storm. Autopsy reports from COVID-19 patients found atrophy in the spleen and secondary lymphoid tissues. Since these organs do not express ACE2, the receptor for COVID-19 infection, Zhang et al. theorize that damage to the immune system was a result of a cytokine storm. Zhang et al. conclude, in agreement with Dr. Roth, that in light of the real possibility of a cytokine storm in critically ill patients, an anti-inflammatory treatment may be necessary, but that “a timely anti-inflammation treatment initiated at the right window of time is of pivotal importance and should be tailored in individual patients to achieve the most favorable effects.” Treatment of cytokine storms by timely administration of an anti-inflammatory agent is further discussed in Kimberly B. Bjugstad's article.

Due to the link between AATD and pneumonia, in one embodiment AAT may be used to treat, control, or prevent pneumonia in patients, either with or without known AATD. A diagnosis of pneumonia may prompt a medical provider to test a patient for AATD. Alternatively, a patient diagnosed with pneumonia may seek genetic testing for AATD through an independent genetic testing company such as 23andMe or geneology.com, a pharmaceutical company supplied test kit or other private methods available.

A method for treating a patient suffering from pneumonia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT may be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended. In the case of treating pneumonia, medical providers may administer AAT more frequently as neutrophil-lymphocyte count ratios (NLCR) can be monitored in these emergency situations by medical providers hour by hour. AAT may be administered in the form of a powder, tablet, or liquid, or AAT may be inhaled, infused, or taken in another manner. For treatment of pneumonia, direct inhalation of AAT into the lungs in varying dosages can be found to be advantageous to the outcome of the patient.

Through the administration of AAT to a patient suffering from pneumonia, the administered AAT inhibits NE that contributes to pneumonia; thus curing, controlling, or reducing the severity of the patient's pneumonia. By using AAT to control NE, pneumonia may be controlled or prevented before it ever develops. In the case of treating pneumonia and/or subsequent cytokine storms, medical providers may administer AAT more frequently as neutrophil-lymphocyte count ratios (NLCR) can be monitored in these emergency situations by medical providers hour by hour to add more efficient control of NE. By using AAT to control NE, pneumonia, and/or potential cytokine storms may be controlled or prevented before the conditions ever develop; therefore, reducing or eliminating the need to use antibiotics to treat the condition of pneumonia, and preventing bacteria from becoming antibiotic resistant.

Psoriatic Arthritis

Excessive neutrophil elastase (“NE”) is associated with psoriatic arthritis and other arthritic conditions. Psoriatic arthritis is a form of arthritis that affects some people who have psoriasis, a condition that features red patches of skin topped with silvery scales. Joint pain, stiffness and swelling are the main signs and symptoms of psoriatic arthritis. They can affect any part of the body, including the fingertips and spine, and can range from relatively mild to severe. In both psoriasis and psoriatic arthritis, disease flare-ups may alternate with periods of remission. No cure for psoriatic arthritis currently exists, and treatment focuses on controlling symptoms and preventing joint damage. Without treatment, psoriatic arthritis may be disabling. Psoriatic arthritis is a chronic, inflammatory disease.

As a powerful inhibitor of NE and inflammation, in one embodiment AAT may be used to treat, control, or prevent psoriatic arthritis in patients, either with or without known AATD. A diagnosis of psoriatic arthritis may prompt a medical provider to test a patient for AATD.

Alternatively, a patient diagnosed with psoriatic arthritis may seek genetic testing for AATD through an independent genetic testing company such as 23andMe or geneology.com, a pharmaceutical company supplied test kit or other private methods available.

A method for treating a patient suffering from psoriatic arthritis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from psoriatic arthritis, inflammation is reduced; thus, the patient's psoriatic arthritis may be cured, controlled, or reduced in severity.

Rheumatoid Arthritis

Excessive neutrophil elastase (“NE”) is associated with rheumatoid arthritis and other arthritic conditions. Rheumatoid arthritis is a chronic, autoimmune, systemic, inflammatory disorder that affects synovial joints, both small and large, in a symmetric pattern. The condition does not cause death but can greatly reduce a patient's quality of life. Joint pain, stiffness and swelling are the main signs and symptoms of rheumatoid arthritis. No cure for rheumatoid arthritis currently exists, and treatment focuses on controlling symptoms, preventing joint damage, and preserve joint function.

As shown by Yap et al, rheumatoid arthritis is strongly associated with various immune cells, and each cell type contributes differently to the condition of rheumatoid arthritis. As a powerful inhibitor of NE and inflammation, in one embodiment AAT may be used to treat, control, or prevent rheumatoid arthritis in patients, either with or without known AATD. A diagnosis of rheumatoid arthritis may prompt a medical provider to test a patient for AATD. Alternatively, a patient diagnosed with rheumatoid arthritis may seek genetic testing for AATD through an independent genetic testing company such as 23andMe or geneology.com, a pharmaceutical company supplied test kit or other private methods available.

A method for treating a patient suffering from rheumatoid arthritis with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from rheumatoid arthritis, inflammation is reduced; thus, the patient's rheumatoid arthritis may be cured, controlled, or reduced in severity.

Scoliosis, Kyphosis, and Related Back Issues

As demonstrated by Morris et al, AATD alleles are associated with joint dislocation and scoliosis in Williams syndrome. Morris et al examined 205 individuals with Williams syndrome for mutations in SERPINA1, the gene that encodes AAT, the inhibitor of elastase. Individuals with classic Williams syndrome deletions and SERPINA1 genotypes PiMS or PiMZ were more likely than those with a SERPINA1 PiMM genotype to have joint dislocation or scoliosis.

Due to the link between AATD and Williams syndrome, kyphosis, scoliosis, and other back issues, in one embodiment AAT may be used to treat, control, or prevent Williams syndrome, scoliosis, and other back issues in patients, either with or without known AATD. A diagnosis of Williams syndrome, kyphosis, scoliosis, or another back issue may prompt a medical provider to test a patient for AATD. Alternatively, a patient diagnosed with Williams syndrome, kyphosis, scoliosis, or another back issue may seek genetic testing for AATD through an independent genetic testing company such as 23andMe or geneology.com, a pharmaceutical company supplied test kit or other private methods available.

A method for treating a patient suffering from Williams syndrome, kyphosis, scoliosis, or another back issue with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from Williams syndrome, kyphosis, scoliosis, or other back issue, the patient's condition is controlled or reduced in severity.

Sickle Cell Anemia

Sickle cell anemia is a condition in which hemoglobin molecules become misshapen and can no longer fit through narrow blood vessels. Sickled red blood cells die after approximately 10 to 20 days rather than the typical 120-day life span of normal red blood cells. Because the red blood cells cannot be replaced quickly enough, patients with sickle cell anemia are chronically short of red blood cells.

In sickle cell anemia, neutrophil extracellular traps (“NETs”) catch misshapen sickle-shaped red blood cells, blocking blood vessels and causing pain. Painful swelling of hands and feet can also occur, and such swelling can be alleviated through the anti-inflammatory action AAT, a powerful inhibitor of neutrophil elastase (“NE”). A link between NETs and several conditions, including sickle cell anemia and other circulatory and coagulation issues, was shown by Brinkmann and Zychlinsky.

Blockage of blood vessels leading to the eyes and retinas can cause blindness in patients suffering from sickle cell anemia. Inhibition of NE using AAT can maintain blood flow to the patient's eyes, preventing blindness.

Organ damage associated with sickle cell anemia can be lessened and eliminated for some patients by preventing the blockage of blood vessels; thus, improving blood flow to vital organs and lessening nerve damage and organ damage.

Through administration of AAT, NETs may be eliminated along with diseases and conditions related to sickle cell anemia, including pain, organ damage, blindness, and other conditions caused or exacerbated by blockages in the microcirculatory system that deprive vital organs and tissues of oxygen. AAT can relieve and treat conditions that can cause high blood pressure or hypertension by reducing NETs that can impair circulation within the circulatory system. By the elimination of NETs, the circulatory system can be maintained in a healthier state. AAT attaches to the excess neutrophils that cause NETs to form, and AAT assists with the safe elimination of NETs from the body.

Oxidative stress contributes to the development of sickle cell anemia. Production of reactive oxygen species is a particularly destructive aspect of oxidative stress. Such species include free radicals and peroxides. Oxidative stress reflects an imbalance between the systemic manifestation of reactive oxygen species and a biological system's ability to detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2- (superoxide radical), OH (hydroxyl radical) and H2O2 (hydrogen peroxide). Further, some reactive oxidative species act as cellular messengers in redox signaling. Thus, oxidative stress can cause disruptions in normal mechanisms of cellular signaling. Hydrogen peroxide is essential for inactivation of AAT, the primary inhibitor of neutrophil elastase. AAT prevents the development of sickle cell anemia through suppression of oxidative stress.

Research has shown a possible correlation between sickle cell anemia and increased risk of death associated with COVID-19.

As a potent inhibitor of NE, NETs, and oxidative stress, in one embodiment AAT may be used to treat, control, or prevent sickle cell anemia and related conditions in patients, either with or without known AATD. A diagnosis of sickle cell anemia may prompt a medical provider to test a patient for AATD. Alternatively, a patient diagnosed with sickle cell anemia may seek genetic testing for AATD through an independent genetic testing company such as 23andMe or geneology.com, a pharmaceutical company supplied test kit or other private methods available.

A method for treating a patient suffering from sickle cell anemia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from sickle cell anemia, the administered AAT neutralizes NE and reduces oxidative stress; thus curing, controlling, or reducing the severity of the patient's sickle cell anemia and therefore potentially assisting in resolving other comorbid conditions in the patient with the addition of a greater amount of circulating AAT in the circulatory system.

Strokes

A stroke is a sudden interruption in the supply of blood to the brain, both thrombotic and embolic. Most strokes are caused by an abrupt blockage of arteries leading to the brain. Hemorrhagic strokes are caused by bleeding into brain tissue when a blood vessel ruptures.

As shown by Moldthan et al, AAT's properties as an endogenous inhibitor of serine proteinases and a primary acute phase protein with potent anti-inflammatory, anti-apoptotic, antimicrobial and cytoprotective activities, could be beneficial in stroke.

As a potent inhibitor of inflammation, in one embodiment AAT may be used to treat, control, or prevent strokes, either with or without known AATD. A diagnosis of stroke may prompt a medical provider to test a patient for AATD. Alternatively, a patient diagnosed with stroke may seek genetic testing for AATD through an independent genetic testing company such as 23andMe or geneology.com, a pharmaceutical company supplied test kit or other private methods available.

A method for treating a patient suffering from stroke with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from a stroke, the administered AAT reduces inflammation; thus curing, controlling, or reducing the severity of the patient's stroke and reducing or preventing tissue damage in the brain.

Surgical Uses

AAT has pre-surgical, post-surgical, and injury treatment applications where AAT can be used to inhibit excessive serine protease activity. Serine protease activity is needed for bacterial or virus control. When bacterial or virus control is not necessary, excessive serine protease activity and neutrophils result in inflammation that must be neutralized or controlled. By controlling and maintaining a balance of neutrophils and AAT, inflammation can be reduced.

Pre-surgical, post-surgical, and injury treatment applications that may benefit from administration of AAT include neurological injuries and neurological disorders (brain injuries, spinal cord injuries, back injuries, etc.) that result in inflammation from excess serine protease and/or neutrophil activity. By reducing the immune system's inflammation response to such conditions, the potential for paralysis and other potential harmful side effects is reduced.

Pre-surgical, post-surgical, and injury treatment applications that may benefit from administration of AAT include injuries to tendons and joints that result in inflammation from excess serine protease and/or neutrophil activity. By reducing the immune system's inflammation response to such conditions, the potential for harmful side effects and/or discomfort of the hip, shoulder, knee, elbow, wrist, hand, and/or foot are reduced.

Pre-surgical, post-surgical, and injury treatment applications that may benefit from administration of AAT include use with bone grafts, hip replacements, shoulder replacements, and knee replacements.

Pre-surgical, post-surgical, and injury treatment applications that may benefit from administration of AAT also include organ transplant surgeries and skin transplant surgeries. AAT may serve as an anti-inflammatory and anti-rejection factor in transplant surgeries.

AAT may provide an alternative treatment option to corticosteroids and/or other anti-inflammation pharmaceuticals for the prevention, limitation, and reduction of inflammation and the neutralization of excess serine protease and/or neutrophil activity. Corticosteroids are known for causing many side effects including: vision problems, swelling, rapid weight gain, shortness of breath, severe depression or unusual thoughts or behaviors, seizures, bloody or tarry stools, hemoptysis, symptoms of pancreatitis (severe pain in the upper abdomen that radiates to the back, nausea, vomiting, and tachycardia), hypokalemia, dangerously high blood pressure, acne, dry skin, thinning skin, bruising or discoloration of skin, insomnia, mood changes; increased sweating, headache, dizziness; nausea, abdominal pain, bloating, slow wound healing, changes in the shape or location of body fat, and degradation of bone, ligament, cartilage, and other tissues. Side effects may also include joint infection, nerve damage, thinning of skin and soft tissue around the injection site, temporary flare of pain and inflammation in the joint; tendon weakening or rupture, thinning of nearby bone (osteoporosis), whitening, or lightening of the skin around the injection site, death of nearby bone (osteonecrosis), and temporary increase in blood sugar. Corticosteroids may also have undesirable drug interactions with: Aspirin, diuretics, blood thinners (Warfarin; Coumadin; Jantoven), Cyclosporine (Restasis; Gengraf; Neoral; Sandimmune), insulin, oral diabetic medications, Ketoconazole (Nizoral), Rifampin (Rifadin), and seizure medications (Phenytoin/Dilantin; Phenobarbital/Luminal/Solfoton).

AAT may be prescribed for managing inflammation in lieu of opioid pain medications which are known to be highly addictive.

Syringomyelia

Syringomyelia is the development of a fluid filled cyst referred to as a syrinx in the spinal cord. Over time, the cyst can become enlarged, damaging the spinal cord, and causing pain, weakness, stiffness, and other symptoms. The majority of syringomyelia cases are caused by a condition in which brain tissue protrudes into the spinal canal, referred to as the Chiari malformation.

AAT may be used to prevent the cyst formation associated with syringomyelia and potentially prevent metastasizing of cancer that may result from cyst formation and inflammation of the spinal canal.

As a potent inhibitor of inflammation, in one embodiment AAT may be used to treat, control, or prevent syringomyelia, either with or without known AATD. A diagnosis of syringomyelia may prompt a medical provider to test a patient for AATD. Alternatively, a patient diagnosed with syringomyelia may seek genetic testing for AATD through an independent genetic testing company such as 23andMe or geneology.com, a pharmaceutical company supplied test kit or other private methods available.

A method for treating a patient suffering from syringomyelia with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from syringomyelia, the administered AAT reduces inflammation; thus curing, controlling, or reducing the severity of the patient's syringomyelia.

Thrombospondin 1 Protein—its Role and Preventing its Destruction or Degradation

Thrombospondin 1 (“tsp1” or “thbs1”) is an adhesive glycoprotein. It can bind to fibrinogen, fibronectin, laminin, type V collagen, and integrins alpha V/beta-1. This protein is a natural inhibitor of neovascularization and tumorigenesis. Tsp1 is known to interact with blood coagulation and anticoagulant factors; thus, tsp1 plays a role in preventing hemophiliac type bleeding and spontaneous type bleeding situations.

Tsp1 protects lung, breast, and other tissues throughout the body by preventing potential cancer cells from developing, by preventing existing cancer cells from maturing, and by preventing existing tumors from metastasizing.

Neutrophils are white blood cells that migrate to the site of an infection in the body to begin killing the invading microbes. However, neutrophils release enzymes such as Neutrophil Elastase (“NE”), Cathepsin G (“CG”), Proteinase 3, and others that target and destroy tsp1. When bacterial or viral control is not necessary, serine protease activity of neutrophils is generally not needed, and can become harmful to the amount of tsp1 available to do the body's natural work against cancer. By controlling and maintaining a balance of neutrophils with AAT, unnecessary inflammation can be reduced or eliminated, while allowing tsp1 and AAT to prevent potential cancer cells from developing, existing cancer cells from maturing, and existing tumors from metastasizing. The presence of healthy tsp1 cells can greatly diminish the spread of metastasizing cells and can allow a patient a better ability to naturally fight off the spread of cancer. The role tsp1 can play in cancer treatment is described by Martin-Manso in their research paper describing how tsp1 interferes with tumor progression. Additionally, Chang et al demonstrated that overexpression of AAT promoted angiogenesis and cell adhesion through increasing expression of tsp1. In contrast, down-regulation of AAT by short hairpin RNA suppressed cell proliferation, metastasis, and adhesion in human lung adenocarcinoma A549 cells and in the lung tissue of K-ras LA1 lung cancer model mice. These findings strongly suggest that AAT regulation shows promise as an alternative avenue for lung cancer treatment and prevention.

Tsp1 protects against metastasis of cancer cells, especially within the lungs. Neutrophils, especially neutrophil elastase, can destroy both lung tissue in the lungs called alveoli and tsp1. This destruction creates an environment that is favorable to the spread of cancer and illness within the lungs and throughout the entire body.

Due to the nature of the interaction between AAT and tsp1, in one embodiment AAT may be used to treat, control, or prevent the destruction of tsp1 in patients, either with or without known AATD. A diagnosis of cancer or other condition associated with destruction or degradation of tsp1 may prompt a medical provider to test a patient for AATD. Alternatively, such a patient may seek genetic testing for AATD through an independent genetic testing company such as 23andMe or geneology.com, a pharmaceutical company supplied test kit or other private methods available.

A method for treating a patient suffering from cancer or other condition associated with destruction or degradation of tsp1 with AAT begins with the step of determining if the patient is AAT deficient or has lower AAT levels without being AATD. Determining if the patient is AAT deficient or has lower AAT levels without being AATD proceeds as previously described in this disclosure in the section entitled “Determining if a patient is AAT deficient or in the state of having low circulating AAT without being AAT deficient.” If the genotyping assay obtained from determining if the patient is AAT deficient indicates that the patient has a Serpina1 AAT deficient genotype or if the patient's circulating AAT level is less than 100 mg/dL, then AAT is administered to the patient in an amount that results in a circulating AAT level in the range of 100-300 mg/dL. Dosage is calculated such that the sum of the patient's current level of circulating AAT plus administered AAT is between the standard range of MM allele individuals. This range typically is described as being between 100 mg/dL and 300 mg/dL. Previous AATD patients have only been brought up to the approximate lowest level on the average level found in patients having MM genotypes. Administration of AAT should be repeated every 5 to 8 days as the life of AAT is estimated at 4.5 days. One drug producer, Grifols, indicates AAT has a half-life of 156 hours, which is approximately 6.5 days. Physicians may fluctuate on 5 to 8 days between treatments, or every 6 to 7 days for typical treatments when dosages are recommended.

Through the administration of AAT to a patient suffering from cancer, the administered AAT counters excessive NE in the patient's system, allowing tsp1 levels to increase; thus curing, controlling, or reducing the severity of the patient's cancer.

At Home Testing for AATD

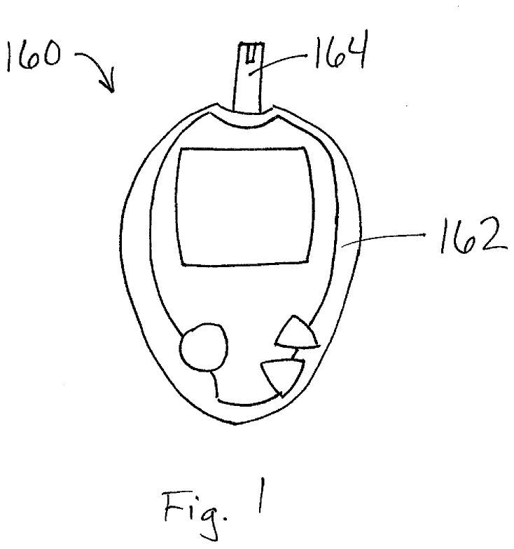

A shown in FIG. 1, a testing device 160 is configured to collect in a collection device 164 a biological sample such as a blood sample from an individual, determine using detection device 162 a genotype and/or phenotype indicative of whether individual has AATD by analyzing the biological sample, and output the result of the analysis. Determining a phenotype indicative of whether the individual has AATD may comprise determining the circulating level of AAT or another substance in the biological sample. The testing device 160 may be used at home or in a medical provider's office. The testing device 160 allows for an individual to determine whether he or she has AAT deficiency at home or in another private setting. By testing privately, an individual can obtain potentially life-saving information without sabotaging future insurance applications and can decide whether to provide test results to third parties such as insurance companies.

Testing device 160 may include, for example, a detection device 162, a collection device or containers 164, instructions, and information describing AAT, AAT deficiency, and treatment options for individuals having AAT deficiency or one or more diseases or disorders associated with AAT deficiency.

Obtaining or having obtained a biological sample from the patient may involve collecting in a collection device 164 a blood sample, urine sample, saliva sample, or other biological sample from which the patient's AAT level can be determined or from which nucleic acids and/or polypeptides can be isolated. The collection device 164 may comprise specimen cups, swabs, glass slides, test tubes, lancets, tubes, syringes, vials, test strips, or any other device known in the art for collecting and/or storing a biological sample from which the patient's AAT level can be determined or from which nucleic acids and/or polypeptides can be isolated.

Determining if the patient is AAT deficient further involves analyzing the collected biological sample using detection device 162 to determine if the patient is AAT deficient. For example, the collected biological sample may be a sample of the patient's blood and determining if the patient is AAT deficient may comprise measuring the circulating AAT level present in the blood sample using a detection device 162 configured to determine the level of AAT circulating in the patient's blood. Alternatively, determining if the patient is AAT deficient may comprise performing or having performed a genotyping assay on the biological sample using a detection device 162 configured to determine the patient's SERPINA1 genotype to determine if the patient is AAT deficient. The genotyping assay may detect which alleles of the SERPINA1 gene are present in the biological sample, indicating whether the patient has a SERPINA1 genotype indicative of AATD. The genotyping assay may detect if the patient has an AAT deficient genotype and/or phenotype directly or may detect characteristic mRNA of the polymorphic gene or its polypeptide expression product. A detection device 162 suitable for use in the methods and devices of the present invention include an at home testing device 160 or any of those known in the art such as polynucleotides used in amplification, sequencing, and single nucleotide polymorphism (SNP) detection techniques, Invader® assays (Third Wave Technologies, Inc.), Taqman® assays (Applied Biosystems, Inc.), gene chip assays (such as those available from Affymetrix, Inc. and Roche Diagnostics), pyrosequencing, fluorescence resonance energy transfer (FRET)-based cleavage assays, fluorescent polarization, denaturing high performance liquid chromatography (DHPLC), mass spectrometry, and polynucleotides having fluorescent or radiological tags used in amplification and sequencing.

Detection means may detect the circulating AAT level of the biological sample. Alternatively, detection means may perform a genotyping assay to determine what alleles of the SERPINA1 gene are present in the biological sample.

Testing device 160 may determine if the individual has AATD by determining the concentration of AAT circulating in the patient's bloodstream. Most hospital laboratories report serum AAT levels in milligrams per deciliter (mg/dL) with a reference range of approximately 100-300 mg/dL. Levels less than 80 mg/dL suggest a significant risk for lung disease. In addition to testing directly for AAT, AATD may be detected by testing device 160 by measuring circulating levels of trypsin, neutrophil elastase, inhibitors, and other substances that are inhibited or moderated by AAT. A testing device 160 for determining circulating levels of AAT or another substance in the patient's blood may use light, electricity, or chemical means for detecting AAT or the other substance.

Testing device 160 may be used to determine information regarding the applicant's unique genetic profile, including whether the applicant has a genetic predisposition for a disease or is a carrier of a disease. Genetic information may include whether the applicant has a genetic predisposition to or is a carrier of AAT deficiency, lung cancer, breast cancer, prostate cancer, other cancers, or other diseases or disorders.

The methods described in this disclosure may have many benefits and advantages including, but not limited to treating and preventing diseases. As AATD often leads to the development of emphysema, lung cancer, and other serious health conditions, knowledge of AAT deficiency can prompt the applicant to seek treatment or preventive care. Using AAT, harsh side effects of many treatments can be avoided, such as the many side effects of radiation and chemotherapy. Through early treatment or prevention of conditions caused by AAT deficiency, many lives can be saved, and quality of life can be vastly improved. These and other benefits and advantages of the methods are apparent from the specification and claims.

### REFERENCE NUMERALS

160—testing device (for at home testing for alpha-1 antitrypsin deficiency)
162—detection device
164—collection device
200—method for treating a condition
210—method step: obtain a sample
220—method step: analyze the sample
230—method step: administer AAT

### REFERENCES

A D Dwarakanath, L G Yu, C Brookes, D Pryce, J M Rhodes, ‘Sticky’ neutrophils, pathergic arthritis, and response to heparin in pyoderma gangrenosum complicating ulcerative colitis, Gut, vol. 37, pp. 585-588, 1995
A. Greco, A. Gallo, M. Fusconi, G. Magliulo, R. Turchetta, C. Marinelli, G. F. Macri, A. De Virgilio, M. de Vincentiis, Cogan's syndrome: An autoimmune inner ear disease, Autoimmune Rev (2012), http://dx.doi.org/10.1016/j.autrev.2012.07.012
A. Kovtun, D. A. C. Messerer, K. Scharffetter-Kochanek, M. Huber-Lang, and A. Ignatius, Neutrophils in Tissue Trauma of the Skin, Bone, and Lung: Two Sides of the Same Coin, Journal of Immunology Research, vol. 2018, article ID 8173983, 12 pages, 23 Apr. 2018
A. Lauhio, T. Salo, Y. Ding, Y. T. Konttinen, D. Nordstrom, H. Tschesche, J. Lahdevirta, L. M. Golub, T. Sorsa, (NSAID) in acute reactive arthritis, Clinical & Experimental Immunology, vol. 98 iss. 1, October 1994
Adam Blaisdell, Amandine Crequer, Devin Columbus, Takiko Daikoku, Khush Mittal, Sudhanshu K. Dey, Adrian Erlebacher, Neutrophils Oppose Uterine Epithelial Carcinogenesis via Debridement of Hypoxic Tumor Cells, Cancer Cell, vol. 28 iss. 6, pp. 785-799, 14 Dec. 2015
Ai-Sheng Ho, Chien-Hsin Chen, Chun-Chia Cheng, Chia-Chi Wang, Hua-Ching Lin, Tsai-Yueh Luo, Gi-Shih Lien, and Jungshan Chang, Neutrophil elastase as a diagnostic marker and therapeutic target in colorectal cancers, Oncotarget, vol. 5(2) pp. 473-480, January 2014
A K Bisgaard, G Pihl-Jensen, J L Frederiksen, The neutrophil-to-lymphocyte ratio as disease activity marker in multiple sclerosis and optic neuritis, Multiple Sclerosis and Related Disorders, vol. 18, pp. 213-217, 1 Nov. 2017
Andjelo Beletic, Aleksandra Dudvarski-Ilic, Branislava Milenkovic, Ljudmila Nagorni-Obradovic, Mila Ljujic, Valentina Djordjevic, Dusko Mirkovic, Dragica Radojkovic, Nada Majkic-Singh, Is an integrative laboratory algorithm more effective in detecting alpha-1-antitrypsin deficiency in patients with premature chronic obstructive pulmonary disease than AAT concentration based screening approach? Biochemia Medica, vol. 24(2), pp. 293-298, 2014
Andrea Nozza, POEMS SYNDROME: an Update, Mediterr J Hematol Infect Dis, vol. 9(1), pub. online 1 Sep. 2017
Anette Breindl, Neutrophil ‘traps’ help tumor cells metastasize, BioWorld, 24 Oct. 2016
Bart W. Bardoel, Elaine F. Kenny, Gabriel Sollberger, Arturo Zychlinsky, The Balancing Act of Neutrophils, Cell Host & Microbe Review, vol. 15, pp. 526-536, 14 May 2014
Benoit Aubé, Sébastien A. Lévesque, Alexandre Paré, Émilie Chamma, Hania Kébir, Roser Gorina, Marc-André Lécuyer, Jorge I. Alvarez, Yves De Koninck, Britta Engelhardt, Alexandre Prat, Daniel Côté and Steve Lacroix, J Immunol, vol. 193(5), pp. 2438-2454, 1 Sep. 2014
Bernard J. Canzoneri, David F. Lewis, Lynn Groome, Yuping Wang, Increased Neutrophil Numbers Account for Leukocytosis in Women with Preeclampsia, Am J Perinatol, vol. 26(10), pp. 729-732, November 2009
Bjugstad, Kimberly B, How to Treat a Cytokine Storm, https://www.endocrineweb.com/professional/endocrinology/how-treat-cytokine-storm, printed Oct. 26, 2020.
Blanco I, Lara B, de Serres F, Efficacy of alpha1-antitrypsin augmentation therapy in conditions other than pulmonary emphysema, Orphanet J Rare Dis, vol. 6:14, 12 Apr. 2011
Blayne A. Sayed, Alison L. Christy, Margaret E. Walker, and Melissa A. Brown, Meningeal Mast Cells Affect Early T Cell Central Nervous System Infiltration and Blood-Brain Barrier Integrity through TNF: A Role for Neutrophil Recruitment?, J Immunol, vol. 184, pp. 6891-6900, pub. online 19 May 2010
Braga P. C., Dal Sasso M., Culici M., Bianchi T., Bordoni L., Marabini L., Anti-Inflammatory Activity of Thymol: Inhibitory Effect on the Release of Human Neutrophil Elastase, Pharmacology, vol. 77, pp. 130-136, 2006
Brice Korkmaz, Marshall S. Horwitz, Dieter E. Jenne, Francis Gauthier, Neutrophil Elastase, Proteinase 3, and Cathepsin G as Therapeutic Targets in Human Diseases, Pharmacological Reviews, vol. 62(4), pp. 726-759, December 2010
Celine Renoux, Marie-Francoise Odou, Guillaume Tasato, Jordan Teoli, Norman Abbou, Christine Lombard, Farid Zerimech, Nicole Poerchet, Colette Chapuis Cellier, Malika Balduyck, Philippe Joly, Description of 22 new alpha-1 antitrypsin genetic variants, Journal of Rare Diseases, vol. 13:161, 2018
Christian W. Keller, Jens Schmidt, Jan D. Lünemann, Immune and myo degenerative pathomechanisms in inclusion body myositis, Ann Clin Transl Neurol, vol. 4(6), pp. 422-445, pub. Online 16 May 2017
Clark P, Boswell F, Greer I A, The neutrophil and preeclampsia, Semin Reprod Endocrinol, vol. 16(1), pp. 57-64, 1998
Clemmensen S N, Jacobsen L C, Rørvig S, Askaa B, Christenson K, Iversen M, Jorgensen M H, Larsen M T, van Deurs B, Ostergaard O, Heegaard N H, Cowland J B, Borregaard N., Alpha-1-antitrypsin is produced by human neutrophil granulocytes and their precursors and liberated during granule exocytosis, Eur J Haematol, vol. 86(6), pp. 517-30, June 2011
Cormac McCarthy, Emer P. Reeves, Noel G. McElvaney, The Role of Neutrophils in Alpha-1 Antitrypsin Deficiency, Ann Am Thorac Soc, vol. 13, supplement 4, pp 5297-5304, August 2016.
Courtney E. Leik, Scott W. Walsh, Neutrophils Infiltrate Resistance-Sized Vessels of Subcutaneous Fat in Women With Preeclampsia, Hypertension vol. 44, pp. 72-77, 2004”
Deepthi Karukayil, Anuradha Sunil, Archana Mukunda, Ashik Bin Basheer, Parry-Romberg syndrome: A case report and an insight in to the advances in its pathophysiology and treatment, Indian Journal of Oral Sciences, vol. 4 iss. 3, pp. 130-134, September-December 2013
Deolinda Silva, Maria João Oliveira, Miguel Guimarães, Ricardo Lima, Silvia Gomes, Susana Seixas, Alpha-1-antitrypsin (SERPINA1) mutation spectrum: Three novel variants and haplotype characterization of rare deficiency alleles identified in Portugal, Respiratory Medicine, vol. 116, pp. 8-18, 1 Jul. 2016
Dieter E. Jenne, Structure of the Azurocidin, Proteinase 3, and Neutrophil Elastase Genes: Implications for Inflammation and Vasculitis, American Journal of Respiratory and Critical Care Medicine, vol. 150 iss. 6 pt. 2, 15 Feb. 1994
Donghui Shen, Fengna Chu, Yue Lang, Yunlong Geng, Xiangyu Zheng, Jie Zhu, Kangding Liu, Beneficial or Harmful Role of Macrophages in Guillain-Barré Syndrome and Experimental Autoimmune Neuritis, Mediators of Inflammation vol. 2018 article ID 4286364
Edaire Cheng, Rhonda F. Souza, Stuart Jon Spechler, Eosinophilic Esophagitis: Interactions with Gastroesophageal Reflux Disease, Gastroenterol Clin North Am vol. 43(2) pp. 243-256, Mar. 24, 2014
Elsa Bronze-da-Rocha and Alice Santos-Silva, Neutrophil Elastase Inhibitors and Chronic Kidney Disease, International Journal of Biological Sciences, vol. 14(10), pp. 1343-1360, 2018
Emiliano Trias, Peter H. King, Ying Si, Yuri Kwon, Valentina Varela, Sofia Ibarburu, Mariangeles Kovacs, Ivan C. Moura, Joseph S. Beckman, Olivier Hermine, and Luis Barbeito, Mast cells and neutrophils mediate peripheral motor pathway degeneration in ALS, JCI Insight. vol. 3(19), article ID e123249, pub. online 4 Oct. 2018
Estelle Hau, Marie-Dominique Vignon Pennamen, Maxime Battistella, Anne Saussine, Maud Bergis, Benedicte Cavelier-Balloy, Michel Janier, Florence Cordoliani, Martine Bagot, Michel Rybojad, Jean-David Bouaziz, Neutrophilic Skin Lesions in Autoimmune Connective Tissue Diseases, Medicine (Baltimore), vol. 93(29), pp. e346, published online 2 Dec. 2014
Eva Polverino, Edmundo Rosales-Mayor, Glenn E. Dale, Klaus Dembowsky, Antoni Torres, The Role of Neutrophil Elastase Inhibitors in Lung Diseases, Chest Journal, vol. 152 iss. 2, pp. 249-262, pub. online 22 Apr. 2017
Garcia-Orad A., Arizti P., Durán L., Urcelay B., de Pancorbo M. M., Alpha-1-Antitrypsin Phenotypes among Breast Cancer Patients in the Basque Population, Hum Hered, vol. 44 no. 4, pp. 203-208, 1994
Gautam Sule, Levi F. Mazza, Nayef M. Kazzaz, Srilakshmi Yalavarthi, He Meng, Jason S. Knight, Inhibition of Neutrophil Elastase Protects Against Glomerulonephritis and Thrombosis in a Mouse Model of Lupus, American College of Rheumatology Meeting Abstracts, 18 Sep. 2017
Gina S. Garcia-Romo, Simone Caielli, Barbara Vega, John Connolly, Florence Allantaz, Zhaohui Xu, Marilynn Punaro, Jeanine Baisch, Cristiana Guiducci, Robert L. Coffman, Franck J. Barrat, Jacques Banchereau, Virginia Pascual, Netting Neutrophils Are Major Inducers of Type I IFN Production in Pediatric Systemic Lupus Erythematosus, Science Translational Medicine, vol. 3 iss. 73, pp. 73ra20, 9 Mar. 2011
Gouni-Berthold I, Baumeister B, Wegel E, Berthold H K, Vetter H, Schmidt C, Neutrophil-elastase in chronic inflammatory bowel disease: a marker of disease activity?, Hepatogastroenterology, vol. 46(28), pp. 2315-20, July-August 1999
H. Golan, I. Kashtuzki, M. Hallak, Y. Sorokin, and M. Huleihel, Maternal Hypoxia During Pregnancy Induces Fetal Neurodevelopmental Brain Damage: Partial Protection by Magnesium Sulfate, Journal of Neuroscience Research, vol. 78, pp. 430-441, published online 7 Sep. 2004
H. M. Nielsen, L. Minthon, E. Londos, K. Blennow, E. Miranda, J. Perez, D. C. Crowther, D. A. Lomas, S. M. Janciauskiene, Plasma and CSF serpins in Alzheimer disease and dementia with Lewy bodies, Neurology, vol. 69:1-1, publ. online 29 Aug. 2007
Ha Y J, Hur J, Go D J, Kang E H, Park J K, Lee E Y, Shin K, Lee E B, Song Y W, Lee Y J, Baseline peripheral blood neutrophil-to-lymphocyte ratio could predict survival in patients with adult polymyositis and dermatomyositis: A retrospective observational study, PLoS One, vol. 13(1), 2 Jan. 2018
Hans A. Cats, Jan Willem Cohen Tervaert, Roelie van Wijk, Piet C. Limburg, Cees G. M. Kallenberg, Anti-Neutrophil Cytoplasmic Antibodies in Giant Cell Arteritis and Polymyalgia Rheumatica, ANCA-Associated Vasculitides pp. 363-366, 1993
H E Jasin and J D Taurog, Mechanisms of disruption of the articular cartilage surface in inflammation. Neutrophil elastase increases availability of collagen type II epitopes for binding with antibody on the surface of articular cartilage, J Clin Invest, vol. 87(5), pp. 1531-1536, May 1991
Hisanori Umehara, Akio Nakajima, Takuji Nakamura, Takafumi Kawanami, Masao Tanaka, Lingli Dong, Mitsuhiro Kawano, IgG4-related disease and its pathogenesis—cross-talk between innate and acquired immunity, Int Immunol, vol. 26(11), pp. 585-595, pub. online 14 Jul. 2014
Hokari, Ryota, Miura, Soichiro, Neutrophil elastase in colitis: more than a marker of disease activity?, Journal of Gastroenterology, vol. 41 (4), pp. 395-396, 1 Jan. 2006
Hoss D M, Shea C R, Grant-Kels J M, Neutrophilic spongiosis in pemphigus, Arch Dermatol, vol. 132(3), pp. 315-318, March 1996
Huong L. Moldthan, Aaron C. Hirko, Jeffrey S. Thinschmidt, Maria Grant, Zhimin Li, Joanna Peris, Yuanqing Lu, Ahmed Elshikha, Michael A. King, Jeffrey A. Hughes, Sihong Song, Alpha 1-Antitrypsin Therapy Mitigated Ischemic Stroke Damage in Rats, J Stroke Cerebrovasc Dis, vol. 23(5), pp. e355-e363, May-June 2014
Irina Lerman, Maria de la Luz Garcia-Hernandez, Javier Rangel-Moreno, Luis Chiriboga, Chunliu Pan, Kent L. Nastiuk, John J. Krolewski, Aritro Sen and Stephen R. Hammes, Infiltrating Myeloid Cells Exert Protumorigenic Actions via Neutrophil Elastase, Mol Cancer Res, vol. 15(9) pp. 1138-1152, September 2017
Iwata K and Mizuno Y, A case of polymyalgia rheumatica following influenza B infection, International Journal of General Medicine, vol. 8, pp. 345-347, 21 Oct. 2015
Izaguirre Anariba, Lanza, Lessnau, Alpha1-Antitrypsin Deficiency Workup, Medscape, pub. 26 Jul. 2019
Izumi G, Koga K, Takamura M, Makabe T, Satake E, Takeuchi A, Taguchi A, Urata Y, Fujii T, Osuga Y, Involvement of immune cells in the pathogenesis of endometriosis, J Obstet Gynaecol Res, vol. 44(2) pp. 191-198, February 2018
J. Fernando Arevalo, Reinaldo A. Garcia, Hassan A. Al-Dhibi, Juan G. Sanchez, Luis Suarez-Tata, Update on Sympathetic Ophthalmia, Middle East Afr J Ophthamol, vol. 19(1), pp. 13-21, January-March 2012
James K Stoller, M D, M S, Felicitas L Lacbawan, M D, and Loutfi S Aboussouan, M D., Alpha-1 Antitrypsin Deficiency, GeneReviews [Internet], Oct. 27, 2006
Jean Donadieu, Blandine Beaupain, Odile Fenneteau, Christine Bellanne-Chantelot, Congenital neutropenia in the era of genomics: classification, diagnosis, and natural history, British Journal of Haematology, vol. 179, pp. 557-574, 2017
Jennifer Huizen, What are neutrophils and what do they do?, Medical News Today, 10 Jan. 2020, printed 2 Apr. 2020, 9 pages
Jodi C. McDaniel, Sashwati Roy, Traci A Wilgus, Neutrophil activity in chronic venous leg ulcers—A target for therapy?, Wound Repair Regen, vol. 21(3), pp. 339-351, May-June 2013
John J. Hooks, Mark O. M. Tso, Barbara Detrick, Retinopathies Associated with Antiretinal Antibodies, Clinical and Diagnostic Laboratory Immunology vol. 8 No. 5 pp. 853-858, September 2001
Joice S L, Mydeen F, Couraud P O, Weksler B B, Romero I A, Fraser P A, Easton A S, Modulation of blood-brain barrier permeability by neutrophils: in vitro and in vivo studies, Brain Res, vol. 1298, pp. 13-23, 17 Nov. 2009
Jordon Dunham, Reinofke van de Vis, Jan Bauer, Jacquelie Wubben, Nikki van Driel, Jon D. Laman, Bert A., Yolanda S. Kap, Severe oxidative stress in an acute inflammatory demyelinating model in the rhesus monkey, PLoS ONE vol. 12(11), Nov. 14, 2017
Jose Angel Nicolas-Avila, Jose M. Adrover, Andres Hidalgo, Neutrophils in Homeostasis, Immunity, and Cancer, Immunity Review, vol. 46, pp. 15-28, 17 Jan. 2017
Justyna Gornowicz-Porowska, Monika Bowszyc-Dmochowska, Agnieszka Seraszek-Jaros, Elżbieta Kaczmarek, Pawel Pietkiewicz, and Marian Dmochowsk, Cutaneous expressions of interleukin-6 and neutrophil elastase as well as levels of serum IgA antibodies to gliadin nonapeptides, tissue transglutaminase and epidermal transglutaminase: implications for both autoimmunity and autoinflammation involvement in dermatitis herpetiformis, Cent Eur J. Immunol. vol. 39(3) pp. 331-337, Oct. 14, 2014
Khawaja Khalid Shoaib, Mooren's ulcer or peripheral ulcerative keratitis, British Journal of Opthalmology, vol. 96 iss. 8, pp. 1146-1147, 2 Jun. 2012
Kuromitsu S, Yokota H, Hiramoto M, Morita S, Mita H, Yamada T, Increased concentration of neutrophil elastase in urine from patients with interstitial cystitis, Scand J Urol Nephrol, vol. 42(5), pp. 455-61, 2008
Kvietys, P. R., and Carter, P. R., Role of neutrophilic elastase in ethanol induced injury to the gastric mucosa, FASEB Journal, 26 Feb. 1990
L Ottonello, M Cutolo, G Frumento, N Arduino, M Vertolotto, M Mancini, E Sottofattori, F Dallegri, Synovial fluid from patients with rheumatoid arthritis inhibits neutrophil apoptosis: role of adenosine and proinflammatory cytokines, Rheumatology, vol. 41 iss. 11, pp. 1249-1260, November 2002
Laidlaw, Stuart & Wagner, Bart & Reilly, John. (2003). Neutrophil inclusions in cryoglobulinemia. British journal of haematology, vol. 122(3):344, September 2003
Lapins J, Asman B, Gustafsson A, Bergstrom K, Emtestam L., Neutrophil-related host response in hidradenitis suppurativa: a pilot study in patients with inactive disease, Acta Derm Venereol vol. 81(2) pp. 96-99, May 2001
Lisa A. Drage, Mark D. P. Davis, Fernando de Castro, Virginia Van Keulen, Ellen A. Weiss, Gerald J. Gleich, Kristin M. Leiferman, Evidence for pathogenic involvement of eosinophils and neutrophils in Churg-Strauss syndrome, Journal of the American Academy of Dermatology vol. 47 iss. 2 pp. 209-216, August 2002
Louise W. Treffers, Ida H. Hiemstra, Taco W. Kujipers, Timo K van den Bert, Hanke L. Matlung, Neutrophils in cancer, Immunological Reviews, vol. 273 iss. 1, Aug. 25, 2016
Mangalmurti, Nilam and Hunter, Christopher A., Cytokine Storms: Understanding COVID-19, Immunity, vol. 53, iss. 1, pp. 19-25, 14 Jul. 2020.
Manoranjan Sahoo, Laura del Barrio, Mark A. Miller, Fabio Re, Neutrophil Elastase Causes Tissue Damage That Decreases Host Tolerance to Lung Infection with Burkholderia Species, PLoS Pathog., vol. 10(8), e1004327, 28 Aug. 2014
Mariana J Kaplan, Neutrophils in the pathogenesis and manifestations of SLE, Nat Rev Rheumatol., vol. 7(12), pp. 691-699, 1 Dec. 2012
Mariana J Kaplan, Role of neutrophils in systemic autoimmune diseases, Arthritis Research & Therapy, vol. 15:219, 2013
Martin-Manso G, Galli S, Ridnour L A, Tsokos M, Wink D A, Roberts D D. Thrombospondin 1 promotes tumor macrophage recruitment and enhances tumor cell cytotoxicity of differentiated U937 cells. Cancer Res 68: 7090-7099, 2008.
Mary Thoesen Coleman and Douglas A. Rund, Nonobstetric conditions causing hypoxia during pregnancy: Asthma and epilepsy, American Journal of Obstetrics and Gynecology, vol. 177 iss. 1, pp. 1-7, July 1997
Matsuse H., Yanagihara K., Mukae H., Tanaka K., Nakazato M., Kohno S., Association of plasma neutrophil elastase levels with other inflammatory mediators and clinical features in adult patients with moderate and severe pneumonia, Respir Med, vol. 101(7), pp. 1521-8, February 2007.
McFarlane D, Hill K, Anton J, Neutrophil function in healthy aged horses and horses with pituitary dysfunction, Vet Immunol Immunopathol, vol. 165(3-4), pp. 99-106, 15 Jun. 2015
Mervat M El-Eshmawy, Eman H El-Adawy, Amany A Mousa, Amany E Zeidan, Azza A El-Baiomy, Elham R Abdel-Samie, and Omayma M Saleh, Elevated serum neutrophil elastase is related to prehypertension and airflow limitation in obese women, BMC Womens Health, vol. 11:1, published online 19 Jan. 2011
Michael D Mueller, Dan I Lebovic, Evelyn Garrett, Robert N Taylor, Neutrophils infiltrating the endometrium express vascular endothelial growth factor: potential role in endometrial angiogenesis, Fertility and Sterility, vol. 74 iss. 1, pp. 107-112, July 2000
Michael Horckmans, Larisa Ring, Johan Duchene, Donato Santovito, Maximilian J. Schloss, Maik Drechsler, Christian Weber, Oliver Soehnlein, Sabine Steffens, Neutrophils orchestrate post-myocardial infarction healing by polarizing macrophages towards a reparative phenotype, European Heart Journal, vol. 38, pp. 187-197, 2017
Mikes B, Sinkovits G, Farkas P, Csuka D, Schlammadinger A, Rázsó K, Demeter J, Domján G, Réti M, Prohászka Z, Elevated plasma neutrophil elastase concentration is associated with disease activity in patients with thrombotic thrombocytopenic purpura, Thromb Res vol. 133(4) pp. 616-21, April 2014
Mitrovic S. & Fautrel B, New markers for adult-onset Still's disease, Joint Bone Spine vol. 85(3) pp. 285-293, May 2018
Monica Lucattelli, Barbara Bartalesi, Eleonora Cavarra, Silvia Fineschi, Benedetta Lunghi, Piero A Martorana, Giuseppe Lungarella, Is neutrophil elastase the missing link between emphysema and fibrosis? Evidence from two mouse models, Respiratory Research, vol. 6, 26 Jul. 2005
Morris C A, Pani A M, Mervis C B, Rios C M, Kistler D J, Gregg R G, Alpha 1 antitrypsin deficiency alleles are associated with joint dislocation and scoliosis in Williams syndrome, Am J Med Genet C Semin Med Genet, vol. 154C(2), pp. 299-306, 15 May 2010
Moxon-Emre I and Schlichter L C, Neutrophil depletion reduces blood-brain barrier breakdown, axon injury, and inflammation after intracerebral hemorrhage, J Neuropathol Exp Neurol, vol. 70(3), pp. 218-235, March 2011
Muley, Reid, Botz, Bolcskei, Helyes, McDougall, Neutrophil elastase induces inflammation and pain in mouse knee joints via activation of proteinase-activated receptor-2, Br J Pharmacol vol. 173(4) pp 766-77, February 2016
N. Arecco, C. J. Clarke, F. K. Jones, D. M. Simpson, D. Mason, R. J. Beynon, A. Pisconti, Elastase levels and activity are increased in dystrophic muscle and impair myoblast cell survival, proliferation and differentiation, Scientific Reports, vol. 6, p. 24708, 31 May 2016
Nadia Luca, Tassalapa Daengsuwan, Josep Dalmau, Kevin Jones, Gabrielle deVeber, Jeffrey Kobayashi, Ronald M. Laxer, Susanne M. Benseler, Anti-N-methyl-D-aspartate receptor encephalitis: a newly recognized inflammatory brain disease in children, Arthritis Rheum vol. 63(8) pp. 2516-22, Jul. 8, 2013
NAKATA, Hiroya; YOKOYA, Yukihiro; KAWAI, Jun; KAWAGUCHI, Shinya; HARA, Takeshi; NAKATA, Hidenori; ITOH, Hidekazu; NISHIOKA, Shingo, Relationship of Helicobacter pylori-associated Gastritis and Polymorphonuclear Leukocyte Elastase, Digestive Endoscopy vol. 6(4), Oct. 1, 1994
Nazrul Islam, Ariful Islam, Syed Jamil Abdal, Mohammad Abul Kalam Azad, Abul Khair Ahmedullah, and Syed Atiqul Haq, Eosinophilic Fasciitis: What Matters in Management in a Developing Country—A Case Report with Two and a Half-year Follow-up, J Health Popul Nutr, vol. 30(1) pp. 117-120, March 2012
Nejat Nizam, Pinar Gümüş, Jari Pitkänen, Taina Tervahartiala, Timo Sorsa, Nurcan Buduneli, Serum and Salivary Matrix Metalloproteinases, Neutrophil Elastase, Myeloperoxidase in Patients with Chronic or Aggressive Periodontitis, Inflammation vol. 37, pp. 1771-1778, 2014
Odile Wéra, Patrizio Lancellotti, Cécile Oury, The Dual Role of Neutrophils in Inflammatory Bowel Diseases, J. Clin. Med., vol. 5, pub. online 17 Dec. 2016
Olaf Wiesner, Kimberly A. Russell, Augustine S. Lee, Dieter E. Jenne, Matteo Trimarchi, Gina Gregorini, Ulrich Specks, Antineutrophil cytoplasmic antibodies reacting with human neutrophil elastase as a diagnostic marker for cocaine-induced midline destructive lesions but not autoimmune vasculitis, Arthritis & Rheumatism vol. 50 iss. 9, Sep. 9, 2004
Oliver P. Jones, ORIGIN OF NEUTROPHILS IN PERNICIOUS ANEMIA (COOKE'S MACROPOLYCYTES), Arch Intern Med (Chic), vol. 60(6), pp. 1002-1015, December 1937
Peros-Golubicić T, Ivicević A, Bekić A, Alilović M, Tekavec-Trkanjec J, Smojver-Jezek S, Lung lavage neutrophils, neutrophil elastase and albumin in the prognosis of pulmonary sarcoidosis, Coll Antropol, vol. 25(1), pp. 349-55, June 2001
Polańska, Bożena, Turno-Krȩcicka, Anna & Kaźmierowska-Niemczuk, Maria, Assessment of neutrophil elastase plasma levels in children with intermediate uveitis, Central European Journal of Immunology vol. 3. pp. 275-279, 2012
Pranvera Sadiku & Sarah R Walmsley, Hypoxia and the regulation of myeloid cell metabolic imprinting: consequences for the inflammatory response, EMBO Reports 20: e47388, published online 14 Mar. 2019
RICHARD H. DUERR, STEPHAN R. TARGAN, CAROL J. LANDERS, NICHOLAS F. LARUSSO, KAREN L. LINDSAY, RUSSELL H. WIESNER, FERGUS SHANAHAN, Neutrophil Cytoplasmic Antibodies: A Link Between Primary Sclerosing Cholangitis and Ulcerative Colitis, Gastroenterology, vol. 100, pp. 1385-1391, 1991
Robert A. Stockley, The multiple facets of alpha-1-antitrypsin, Ann Transl Med, vol. 3(10), p. 130, June 2015
Robert Grecian, Moira K B Whyte, Sarah R Walmsley, The role of neutrophils in cancer, British Medical Bulletin, vol. 128 iss. 1 pp. 5-14, December 2018
Sabroe R A, Poon E, Orchard G E, Lane D, Francis D M, Barr R M, Black M M, Black A K, Greaves M W, Cutaneous inflammatory cell infiltrate in chronic idiopathic urticaria: comparison of patients with and without anti-FcepsilonRI or anti-IgE autoantibodies, J Allergy Clin Immunol vol. 103(3 pt. 1) pp. 484-93, March 1999
Sadaf Afzal, Maria Recio, Sadat Shamim, Paraneoplastic cerebellar ataxia and the paraneoplastic syndromes, Proc (Bayl Univ Med Cent), vol. 28(2), pp. 217-220, April 2015
Samira Saadoun, Patrick Waters, Claire MacDonald, Anthony Bell, Angela Vincent, A. S. Verkman, Marios Papadopoulos, Neutrophil protease inhibition reduces neuromyelitis optica-immunoglobulin G-induced damage in mouse brain, first published Dec. 5, 2011, https://doi.org/10.1002/ana.22686
Samira Saadoun, Patrick Waters, Claire MacDonald, B. Anthony Bell, Angela Vincent, A. S. Verkman, Marios C. Papadopoulos, Neutrophil Protease Inhibition Reduces Neuromyelitis Optica-Immunoglobulin G-Induced Damage in Mouse Brain, Ann Neurol, vol. 71(3), pp. 323-333, March 2012
Scott D A and Krauss J, Neutrophils in periodontal inflammation, Front Oral Biol, vol. 15, pp. 56-83, 2012
Seiichiro Takeshita, Yoichi Kawamura, Takashi Kanai, Yusuke Yoshida, Yuki Tsujita, Shigeaki Nonoyama, The Role of Neutrophil Activation in the Pathogenesis of Kawasaki Disease, Pediatric Infect Dis, vol. 3 no. 1:1, 4 Jan. 2018
Seth B. Coffelt, Max D. Wellenstein, Karin E. de Visser, Neutrophils in cancer: neutral no more, Nature Reviews Cancer, vol. 16, pp. 431-446, 2016
Seung-Hee Chang, Kyung-Cho Cho, Kyeong-Nam Yu, Seong-Ho Hong, Park Sungjin, Ah Lee, Sanghwa Kim, Kang Jeong Won, Chanhee Chae, Jongsun Park, Kwang pyo kim, Myung-Haing Cho, Alpha 1-antitrypsin activates lung cancer cell survival by acting on cap-dependent protein translation, vesicle-mediated transport, and metastasis, Oncotarget, 2014
Sevuk U, Bilgic A, Altindag R, Baysal E, Yaylak B, Adiyaman M S, Akkaya S, Ay N, Alp V, Value of the neutrophil-to-lymphocyte ratio in predicting post-pericardiotomy syndrome after cardiac surgery, Eur Rev Med Pharmacol Sci, vol. 20(5), pp. 906-911, March 2016
Shingo Ai, Musafumi Kuzuya, Akihisa Iguchi, Neutrophil elastase in pressure ulcer fluid degrades fibronectin in the exudates, Geriatrics & Gerontology International, vol. 4 iss. 3, 10 Aug. 2004
SOHAM CHAUDHARI, NARCISS MOBINI, Linear IgA Bullous Dermatosis A Rare Clinicopathologic Entity with an Unusual Presentation, Journal of Clinical Aesthetic Dermatology, vol. 8 no. 10, pp. 43-46, October 2015
Srilakshmi Yalavarthi, Travis J. Gould, Ashish N. Rao, Levi F. Mazza, Alexandra E. Morris, Carlos Núñez-Álvarez, Diego Hernández-Ramirez, Paula L. Bockenstedt, Patricia C. Liaw, Antonio R. Cabral, and Jason S. Knight, antiphospholipid antibodies promote the release of neutrophil extracellular traps: a new mechanism of thrombosis in the antiphospholipid syndrome, Arthritis Rhematol. vol. 67(11) pp. 2990-3003, November 2015
Sten Gershagen & Sabina Janciauskiene, ELISA for Specific Detection of PiZ-Related al-Antitrypsin Deficiency, Clinical Chemistry, vol. 50 iss. 12, pp. 2407-2410, 1 Dec. 2004
Sylvie Verraes, William Hornebeck, Myriam Polette, Luca Borradori, and Philippe Bernard, Respective Contribution of Neutrophil Elastase and Matrix Metalloproteinase 9 in the Degradation of BP180 (Type XVII Collagen) in Human Bullous Pemphigoid, Journal of Investigative Dermatology vol. 117 iss. 5, pp. 1091-1096, November 2001
Taggart C, Cervantes-Laurean D, Kim G, McElvaney N G, Wehr N, Moss J, Levine R L, Oxidation of either Methionine 351 or Methionine 358 in al-Antitrypsin Causes Loss of Anti-neutrophil Elastase Activity, J Biol Chem, vol. 275(35), pp. 27258-65, 1 Sep. 2000
Takahashi H, Nukiwa T, Yoshimura K, Quick C D, States D J, Holmes M D, Whang-Peng J, Knutsen T, Crystal R G, Structure of the human neutrophil elastase gene, J Biol Chem, vol. 263(29), pp. 14739-47, 15 Oct. 1988
Tierney P, Chan B, Samuel D, Thomas M, Patel K, Neutrophil elastase-alpha 1-antitrypsin in middle ear fluid in chronic otitis media with effusion, Clin Otolaryngol Allied Sci, vol. 20(3), pp. 230-233, June 1995
Tina El Rayes, Ranl Catena, Sharrell Lee, Marcin Stawowczyk, Natasha Joshi, Claudia Fischbach, Charles A. Powell, Andrew J. Dannenberg, Nasser K. Altorki, Dingcheng Gao, Vivek Mittal, Lung inflammation promotes metastasis through neutrophil protease-mediated degradation of Tsp-1, PNAS, vol. 112 (52), pp. 16000-16005; 14 Dec. 2015
Tom Macleod, Rosella Doble, Dennis McGonagle, Christopher W. Wasson, Adewonuola Alase, Martin Stacey, Miriam Wittmann, Neutrophil Elastase-mediated proteolysis activates the anti-inflammatory cytokine IL-36 Receptor antagonist, Sci Rep, vol. 6, p. 24880, pub. online 22 Apr. 2016
Vincent M. Tutino, Kerry E. Poppenberg, Kaiyu Jiang, James N. Jarvis, Yijun Sun, Ashish Sonig, Adnan H. Siddiqui, Kenneth V. Snyder, Elad I. Levy, John Kolega, Hui Meng, Circulating neutrophil transcriptome may reveal intracranial aneurysm signature, PLOS One17 Jan. 2018
Volker Brinkmann and Arturo Zychlinsky, Neutrophil extracellular traps: Is immunity the second function of chromatin?, J Cell Biol, vol. 198(5), pp. 773-783, 3 Sep. 2012
von Wedel-Parlow M, Schrot S, Lemmen J, Treeratanapiboon L, Wegener J, Galla H J, Neutrophils cross the BBB primarily on transcellular pathways: an in vitro study, Brain Res, vol. 1367, pp. 62-76
Warren Stevens, Tiffany Shih, Devin Incerti, Thanh G. N. Ton, Henry C. Lee, Desi Peneva, George A. Macones, Baha M. Sibai, Anupam B. Jena, Short-term costs of preeclampsia to the United States health care system, Reports of Major Impact, vol. 217 iss. 3, pp. 237-248, 1 Sep. 2017
Wenge Liu, Kenji Okajima, Kazunori Murakami, Naoaki Harada, Hirotaka Isobe, Tetsumilrie Irie, Role of neutrophil elastase in stress-induced gastric mucosal injury in rats, Journal of Laboratory and Clinical Medicine, vol. 132 iss. 5, pp. 432-439, November 1998
William Bracamonte-Baran & Daniela Čiháková, Cardiac Autoimmunity: Myocarditis, Adv Exp Med Biol, vol. 1003, pp. 187-221, 1 Jan. 2018
Xinhua Yu, Reza Akbarzadeh, Mario Pieper, Thomas Scholzen, Stefanie Gehrig, Carsten Schultz, Detlef Zillikens, Peter Konig, Frank Petersen, Neutrophil Adhesion Is a Prerequisite for Antibody-Mediated Proteolytic Tissue Damage in Experimental Models of Epidermolysis Bullosa Acquisita, Journal of Investigative Dermatology. https://doi.org/10.1016/j.jid.2018.03.1499
XU HUANG, YANG YU, HUA ZHANG, JIE LIU, YUQIAN SUN, MANLI CHANG, and CAN CUI, Hashimoto encephalopathy associated with hyperthyroidism: A case report, Exp Ther Med vol. 8(2) pp. 515-518, August 2014
Yang D H, Qian M Z, Wei M M, Li J, Yu M M, Lu X M, Yang H, Lin H, Li X, Zhu J Y, Zhang X, The correlation of neutrophil-to-lymphocyte ratio with the presence and activity of myasthenia gravis, Oncotarget, vol. 8(44), pp. 76099-76107, 16 Jun. 2017
Yang-Wuyue Liu, Song Li, Shuang-Shuang Dai, Neutrophils in traumatic brain injury (TBI): friend or foe?, Journal of Neuroinflammation, vol 15:146, 2018
Yao Liu, Jie Fu, Jie Zhang, Ying Wang and Xiaobing Guan, A Case Report on Recurrent Oral Ulcers Associated with Cyclic Neutropenia, Ann Clin Case Rep. vol. 2: 1314, 2017
Yap, Hooi-Yeen, Zi-Yi Tee, Sabrina, Mei-Theng Wong, Magdelyn, Chow, Sook-Khuan, Peh, Suat-Cheng, Teowl, Sin-Yeang, Pathogenic Role of Immune Cells in Rheumatoid Arthritis: Implications in Clinical Treatment and Biomarker Development, Cells, published online 2018 Oct. 9. doi: 10.3390/cells7100161.
YASUYUKI TAOOKA, AKIHIRO MAEDA, KEIKO HIYAMA, SHINICHI ISHIOKA, MICHIO YAMAKIDO, Effects of Neutrophil Elastase Inhibitor on Bleomycin-Induced Pulmonary Fibrosis in Mice, American Journal of Respiratory and Critical Care Medicine, vol. 156 iss. 1, 13 Dec. 1996
Yi-Di Liu, Yue-Ping Zeng, Ya-Nan Wang, Wen-Ling Zhao, Yue-Hua Liu, and Li Li, Pityriasis Lichenoides et Varioliformis Acuta and Psoriasis Vulgaris: Mere Coincidence or a Rare Association?, Chin Med J (Engl), vol. 130(4), pp. 501-502, 20 Feb. 2017
Ying-Qiu Huang, Recent advances in the diagnosis and treatment of primary biliary cholangitis, World J Hepatol, vol. 8(33), pp. 1419-1441, 28 Nov. 2016
Yotam Lior, Assaf Geyra, Eli C. Lewis, Therapeutic compositions and uses of alpha1-antitrypsin: a patent review (2012-2015), Expert opinion on therapeutic patents vol. 26 5, pp. 581-9, 2016
Yu-Mee Kim, Leila Haghighat, Edda Spiekerkoetter, Hirofumi Sawada, Cristina M. Alvira, Lingli Wang, Swati Acharya, Gabriela Rodriguez-Colon, Andrew Orton, Mingming Zhao, and Marlene Rabinovitch, Neutrophil Elastase Is Produced by Pulmonary Artery Smooth Muscle Cells and Is Linked to Neointimal Lesions, Am J Pathol, vol. 179(3), pp. 1560-1572, September 2011
Yumiko Hamano, Kina Kase, Ayano Tetsuka, Mari Dias, Kenichi Nakamura, Toru Sasaki, Makoto Ito, Surgical Indication of Inner Ear Malformation associated with Bacterial Meningitis, Clin Surg vol. 3, 13 Jul. 2018
Zhang S, Shu X, Tian X, Chen F, Lu X, Wang G, Enhanced formation and impaired degradation of neutrophil extracellular traps in dermatomyositis and polymyositis: a potential contributor to interstitial lung disease complications, Clin Exp Immunol, vol. 177(1), pp. 134-41, July 2014.
Zhang W, Zhao Y, Zhang F, et al., The use of anti-inflammatory drugs in the treatment of people with severe coronavirus disease 2019 (COVID-19): The Perspectives of clinical immunologists from China, Clin Immunol. 2020; 214:108393.
Zhi Liu, Steven D. Shapiro, Xiaoye Zhou, Sally S. Twining, Robert M. Senior, George J. Giudice, Janet A. Fairley, Luis A. Diaz, A critical role for neutrophil elastase in experimental bullous pemphigoid, J Clinical Investigation vol. 105(1) pp. 113-123, Jan. 1, 2000.
Zhifu Sun, Ping Yang, Role of imbalance between neutrophil elastase and al-antitrypsin in cancer development and progression, The Lancet Oncology vol. 5 iss. 3 pp. 182-190, March

What is claimed:

1. A method of treating a patient with alpha-1 antitrypsin, wherein the patient is suffering from a condition, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is in the range of 100-300 mg/dL.

2. The method of claim 1 wherein performing or having performed an analysis of the biological sample comprises performing or having performed a genotyping assay on the biological sample to determine if the patient has a Serpina1 alpha-1 antitrypsin deficient genotype.

3. The method of claim 2 further comprising measuring or having measured the level of circulating alpha-1 antitrypsin in the biological sample.

4. The method of claim 1 wherein performing or having performed an analysis of the biological sample comprises measuring or having measured the level of circulating alpha-1 antitrypsin in the biological sample.

5. The method of claim 1 further comprising repeating the step of internally administering alpha-1 antitrypsin to the patient such that the patient's total alpha-1 antitrypsin level is maintained in the range of 100-300 mg/dL.

6. A method of treating a patient with alpha-1 antitrypsin, wherein the patient is suffering from pneumonia, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is in the range of 100-300 mg/dL.

7. The method of claim 6 wherein performing or having performed an analysis of the biological sample comprises performing or having performed a genotyping assay on the biological sample to determine if the patient has a Serpina1 alpha-1 antitrypsin deficient genotype.

8. The method of claim 7 further comprising measuring or having measured the level of circulating alpha-1 antitrypsin in the biological sample.

9. The method of claim 6 wherein performing or having performed an analysis of the biological sample comprises measuring or having measured the level of circulating alpha-1 antitrypsin in the biological sample.

10. The method of claim 6 further comprising monitoring the patient's alpha-1 antitrypsin level and repeating the step of internally administering alpha-1 antitrypsin to the patient such that the patient's total alpha-1 antitrypsin level is maintained in the range of 100-300 mg/dL.

11. A method of preventing destruction or degradation of thrombospondin 1 protein in body of a patient, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is in the range of 100-300 mg/dL.

12. The method of claim 11 wherein performing or having performed an analysis of the biological sample comprises performing or having performed a genotyping assay on the biological sample to determine if the patient has a Serpina1 alpha-1 antitrypsin deficient genotype.

13. The method of claim 12 further comprising measuring or having measured the level of circulating alpha-1 antitrypsin in the biological sample.

14. The method of claim 11 wherein performing or having performed an analysis of the biological sample comprises measuring or having measured the level of circulating alpha-1 antitrypsin in the biological sample.

15. The method of claim 11 further comprising repeating the step of internally administering alpha-1 antitrypsin to the patient such that the patient's total alpha-1 antitrypsin level is maintained in the range of 100-300 mg/dL.

16. A method of preventing destruction, damage, or demyelination of cells by excess neutrophils in a body of a patient, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is maintained in the range of 100-300 mg/dL.

17. The method of claim 16 wherein performing or having performed an analysis of the biological sample comprises performing or having performed a genotyping assay on the biological sample to determine if the patient has a Serpina1 alpha-1 antitrypsin deficient genotype.

18. The method of claim 17 further comprising measuring or having measured the level of circulating alpha-1 antitrypsin in the biological sample.

19. The method of claim 16 wherein performing or having performed an analysis of the biological sample comprises measuring or having measured the level of circulating alpha-1 antitrypsin in the biological sample.

20. The method of claim 16 further comprising repeating the step of internally administering alpha-1 antitrypsin to the patient such that the patient's total alpha-1 antitrypsin level is maintained in the range of 100-300 mg/dL.

21. A method of reducing inflammation and reducing pain in a body of a patient, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is in the range of 100-300 mg/dL.

22. The method of claim 21 wherein performing or having performed an analysis of the biological sample comprises performing or having performed a genotyping assay on the biological sample to determine if the patient has a Serpina1 alpha-1 antitrypsin deficient genotype.

23. The method of claim 22 further comprising measuring or having measured the level of circulating alpha-1 antitrypsin in the biological sample.

24. The method of claim 21 wherein performing or having performed an analysis of the biological sample comprises measuring or having measured the level of circulating alpha-1 antitrypsin in the biological sample.

25. The method of claim 21 further comprising repeating the step of internally administering alpha-1 antitrypsin to the patient such that the patient's total alpha-1 antitrypsin level is maintained in the range of 100-300 mg/dL.

26. A method of preventing metastasis of cancer in a body of a patient diagnosed with cancer, the method comprising the steps of:
determining if the patient is alpha-1 antitrypsin deficient by:
obtaining or having obtained a biological sample from the patient, and
performing or having performed an analysis of the biological sample; and
if the patient is alpha-1 antitrypsin deficient, then internally administering alpha-1 antitrypsin to the patient such that the patient's total alpha-1 antitrypsin level is in the range of 100-300 mg/dL.

27. The method of claim 26 wherein performing or having performed an analysis of the biological sample comprises performing or having performed a genotyping assay on the biological sample to determine if the patient has a Serpina1 alpha-1 antitrypsin deficient genotype.

28. The method of claim 27 further comprising measuring or having measured the level of circulating alpha-1 antitrypsin in the biological sample.

29. The method of claim 26 wherein performing or having performed an analysis of the biological sample comprises measuring or having measured the level of circulating alpha-1 antitrypsin in the biological sample.

30. The method of claim 26 further comprising repeating the step of internally administering alpha-1 antitrypsin to the patient such that the patient's total alpha-1 antitrypsin level is maintained in the range of 100-300 mg/dL.

31. The method of claim 25 further comprising monitoring the patient's alpha-1 antitrypsin level.
